(19) United States
(12) Patent Application Publication
(10) Pub. No.: US 2013/0280212 A1
(43) Pub. Date: Oct. 24, 2013

(54) INHIBITORS OF CYTOCHROME P450

(71) Applicants: Manoj C. Desai, Pleasant Hill, CA (US); Hongtao Liu, Cupertino, CA (US); Lianhong Xu, Palo Alto, CA (US); Hong Ye, Richmond, CA (US)

(72) Inventors: Manoj C. Desai, Pleasant Hill, CA (US); Hongtao Liu, Cupertino, CA (US); Lianhong Xu, Palo Alto, CA (US); Hong Ye, Richmond, CA (US)

(73) Assignee: GILEAD SCIENCES, INC, Foster City, CA (US)

(21) Appl. No.: 13/996,174

(22) PCT Filed: Dec. 20, 2011
(86) PCT No.: PCT/US11/66247

§ 371 Date: Jun. 20, 2013

Related U.S. Application Data
(60) Provisional application No. 61/425,387, filed on Dec. 21, 2010.

(51) Int. Cl. C07D 277/24; A61K 45/06; A61K 31/426

(52) U.S. Cl. CPC C07D 277/24; A61K 31/426; A61K 45/06
USPC 424/85.4; 548/204; 514/365

(57) ABSTRACT

The present application provides for a compound of formula (I), or a pharmaceutically acceptable salt, solvate, and/or ester thereof, compositions containing such compounds, therapeutic methods that include the administration of such compounds, and therapeutic methods that include the administration of such compounds with at least one additional therapeutic agent.

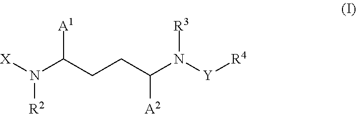

### PRIORITY OF INVENTION

This application claims priority to U.S. Provisional Patent Application No. 61/425,387 filed 21 Dec. 2010. The entire content of this application is hereby incorporated herein by reference.

### FIELD OF THE INVENTION

This application relates generally to compounds and pharmaceutical compositions which improve the pharmacokinetics of a co-administered drug, and methods of improving, the pharmacokinetics of a drug by co-administration of the compounds with the drug.

### BACKGROUND OF THE INVENTION

Oxidative metabolism by cytochrome P450 enzymes is one of the primary mechanisms of drug metabolism. It can be difficult to maintain therapeutically effective blood plasma levels of drugs which are rapidly metabolized by cytochrome P450 enzymes. Accordingly, the blood plasma levels of drugs which are susceptible to cytochrome P450 enzyme degradation can be maintained or enhanced by co-administration of cytochrome P450 inhibitors, thereby improving the pharmacokinetics of the drug.

While certain drugs are known to inhibit cytochrome P450 enzymes, more and/or improved inhibitors for cytochrome P450 monooxygenase are desirable. Particularly, it would be desirable to have cytochrome P450 monooxygenase inhibitors which do not have appreciable biological activity other than cytochrome P450 inhibition. Such inhibitors can be useful for minimizing undesirable biological activity (e.g., side effects). For example, it would be desirable to have P450 monooxygenase inhibitors that lack significant or have a reduced level of protease inhibitor activity. Such inhibitors could be useful for enhancing the effectiveness of antiretroviral drugs, while minimizing the possibility of eliciting viral resistance, especially against protease inhibitors.

### SUMMARY OF THE INVENTION

One aspect of the present application is directed to compounds and pharmaceutical compositions which improve the pharmacokinetics of a co-administered drug. Representative examples of the invention also demonstrated little or no HIV protease inhibition activity.

In one embodiment, the invention provides a compound which is a compound of formula I:

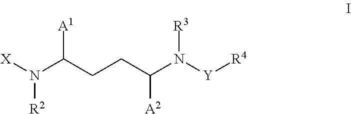

wherein:

A¹ is (C₁-C₆)alkyl, aryl(C₁-C₆)alkyl, heteroaryl(C₁-C₆)alkyl, (C₃-C₆)carbocyclyl(C₁-C₆)alkyl or heterocyclyl(C₁-C₆)alkyl, wherein any (C₁-C₆)alkyl of A¹ is optionally substituted with one or more (e.g. 1, 2, 3, 4 or 5) Z¹ groups and wherein any aryl(C₁-C₆)alkyl, heteroaryl(C₁-C₆)alkyl, (C₃-C₆)carbocyclyl(C₁-C₆)alkyl or heterocyclyl(C₁-C₆)alkyl of A¹ is optionally substituted with one or more (e.g. 1, 2, 3, 4 or 5) Z² groups;

A² is (C₁-C₆)alkyl, aryl(C₁-C₆)alkyl, heteroaryl(C₁-C₆)alkyl, (C₃-C₆)carbocyclyl(C₁-C₆)alkyl or heterocyclyl(C₁-C₆)alkyl, wherein any (C₁-C₆)alkyl of A² is optionally substituted with one or more (e.g. 1, 2, 3, 4 or 5) Z¹ groups and wherein any aryl(C₁-C₆)alkyl, heteroaryl(C₁-C₆)alkyl, (C₃-C₆)carbocyclyl(C₁-C₆)alkyl or heterocyclyl(C₁-C₆)alkyl of A² is optionally substituted with one or more (e.g. 1, 2, 3, 4 or 5) Z² groups;

X is selected from the group consisting of R^{1a}OC(O)—, R^{1b}C(O)—, R^{1a}(R^{a})NC(O)—, R^{1b}S(O)₂— and R^{1a}(R^{a})NS(O)₂—;

Y is —C(O)O— or —C(O)NR^{b}—;

R^{1a} is (C₁-C₈)alkyl, (C₃-C₆)carbocyclyl, aryl or aryl(C₁-C₆)alkyl, wherein any (C₁-C₈)alkyl of R^{1a} is optionally substituted with one or more (e.g. 1, 2, 3, 4 or 5) Z³ groups, and wherein any (C₃-C₆)carbocyclyl, aryl or aryl(C₁-C₆)alkyl of R^{1a} is optionally substituted with one or more (e.g. 1, 2, 3, 4 or 5) Z⁴ groups;

R^{1b} is (C₁-C₈)alkyl, (C₃-C₆)carbocyclyl, aryl, aryl(C₁-C₆)alkyl, heteroaryl, heteroaryl(C₁-C₆)alkyl, heterocyclyl or heterocyclyl(C₁-C₆)alkyl, wherein any (C₁-C₈)alkyl of R^{1b} is optionally substituted with one or more (e.g. 1, 2, 3, 4 or 5) Z³ groups and wherein any (C₃-C₆)carbocyclyl, aryl, aryl(C₁-C₆)alkyl, heteroaryl, heteroaryl(C₁-C₆)alkyl, heterocyclyl or heterocyclyl(C₁-C₆)alkyl of R^{1b} is optionally substituted with one or more (e.g. 1, 2, 3, 4 or 5) Z⁴ groups;

R² is selected from the group consisting of H, (C₁-C₆)alkyl, (C₃-C₆)carbocyclyl, (C₃-C₆)carbocyclyl(C₁-C₆)alkyl- and aryl(C₁-C₆)alkyl;

R³ is selected from the group consisting of H, (C₁-C₆)alkyl, (C₃-C₆)carbocyclyl, (C₃-C₆)carbocyclyl(C₁-C₆)alkyl- and aryl(C₁-C₆)alkyl;

R⁴ is aryl, aryl(C₁-C₆)alkyl, heteroaryl, heteroaryl(C₁-C₆)alkyl, heterocyclyl or heterocyclyl(C₁-C₆)alkyl, wherein any aryl, aryl(C₁-C₆)alkyl, heteroaryl, heteroaryl(C₁-C₆)alkyl, heterocyclyl or heterocyclyl(C₁-C₆)alkyl of R⁴ is optionally substituted with one or more (e.g. 1, 2, 3, 4 or 5) Z⁵ groups;

each Z¹ is independently selected from OH, oxo, halogen, OCF₃, CN, —O(C₁-C₆)alkyl, S(C₁-C₆)alkyl, SO(C₁-C₆)alkyl, SO₂(C₁-C₆)alkyl, —NR^{c}R^{d}, —NRⁱC(O)R^{j} and —NRⁱS(O)₂R^{j};

each Z² is independently selected from OH, oxo, halogen, CF₃, OCF₃, NO₂, CN, (C₁-C₆)alkyl, —O(C₁-C₆)alkyl, S(C₁-C₆)alkyl, SO(C₁-C₆)alkyl, SO₂(C₁-C₆)alkyl, —NR^{c}R^{d}, —NRⁱC(O)R^{j} and —NRⁱS(O)₂R^{j};

each Z³ is independently selected from OH, halogen, CN, —O(C₁-C₆)alkyl, and —NR^{e}R^{f};

each Z⁴ is independently selected from OH, halogen, CF₃, OCF₃, NO₂, CN, (C₁-C₆)alkyl, —O(C₁-C₆)alkyl, aryl and —NR^{e}R^{f}, wherein any aryl of Z⁴ is optionally substituted with one or more (e.g. 1, 2, 3, 4 or 5) groups selected from OH, halogen, —CF₃, —OCF₃, NO₂, CN, (C₁-C₆)alkyl and —O(C₁-C₆)alkyl;

each Z⁵ is independently selected from OH, oxo, halogen, CF₃, OCF₃, NO₂, CN, (C₁-C₆)alkyl, —O(C₁-C₆)alkyl, and —NR^{g}R^{h};

R^{a} is selected from the group consisting of H, (C₁-C₆)alkyl and aryl(C₁-C₆)alkyl;

R^{b} is selected from the group consisting of H, (C₁-C₆)alkyl and aryl(C₁-C₆)alkyl;

R^{c} and R^{d} are each independently selected from H and (C₁-C₆)alkyl; or R^{c} and R^{d} together with the nitrogen to which they are attached form a piperidine, morpholine, piperazine, N-methylpiperazine or pyrrolidine;

R^{e} and R^{f} are each independently selected from H and (C₁-C₆)alkyl; or R^{e} and R^{f} together with the nitrogen to which they are attached form a piperidine, morpholine, piperazine, N-methylpiperazine or pyrrolidine;

R^{g} and R^{h} are each independently selected from H and (C₁-C₆)alkyl; or R^{g} and R^{h} together with the nitrogen to which they are attached form a piperidine, morpholine, piperazine, N-methylpiperazine or pyrrolidine;

Rⁱ is H or (C₁-C₆)alkyl; and

R^{j} is (C₁-C₆)alkyl;

or a salt thereof.

In another embodiment, the invention provides a pharmaceutical composition comprising a compound of formula I, or a pharmaceutically acceptable salt thereof and a pharmaceutically acceptable carrier or excipient.

In another embodiment, the invention provides a pharmaceutical composition comprising: 1) a compound of formula I or pharmaceutically acceptable salt thereof, 2) one or more (e.g. 1, 2, 3 or 4) therapeutic agents, and 3) a pharmaceutically acceptable carrier or excipient.

In another embodiment, the invention provides a method for improving the pharmacokinetics of a therapeutic agent, comprising co-administration to a patient the therapeutic agent and a compound of formula I or a pharmaceutically acceptable salt thereof.

In another embodiment, the invention provides a method for increasing the blood plasma levels of a therapeutic agent, comprising co-administration to a patient the therapeutic agent and a compound of formula I or a pharmaceutically acceptable salt thereof.

In another embodiment, the invention provides a method for inhibiting cytochrome P450 monooxygenase in a patient comprising administering to a patient in need thereof an amount of a compound of formula I, or a pharmaceutically acceptable salt thereof, effective to inhibit cytochrome P450 monooxygenase.

In another embodiment, the invention provides a method for treating a viral infection, (e.g., HIV, HCV) comprising co-administration to a patient in need thereof a therapeutically effective amount of a compound of formula I, or a pharmaceutically acceptable salt thereof, in combination with a therapeutically effective amount of, one or more (e.g. 1, 2, 3, and 4) therapeutic agents which are metabolized by cytochrome P450 monooxygenase, and which are suitable for treating a viral infection (e.g., HIV, HCV).

In another embodiment, the invention provides a combination pharmaceutical agent comprising:

a) a first pharmaceutical composition comprising a compound of formula I, or a pharmaceutically acceptable salt thereof; and

b) a second pharmaceutical composition comprising at least one therapeutically active agent which is metabolized by cytochrome P450 monooxygenase.

In another embodiment, the invention provides a combination pharmaceutical agent comprising:

a) a compound of formula I, or a pharmaceutically acceptable salt thereof; and

b) at least one therapeutically active agent which is metabolized by cytochrome P450 monooxygenase.

In another embodiment the invention provides a compound of formula I, or a pharmaceutically acceptable salt thereof for use in medical therapy.

In another embodiment the invention provides the use of a compound of formula I, or a pharmaceutically acceptable salt thereof for the manufacture of a medicament useful for improving the pharmacokinetics of a therapeutic agent which is metabolized by cytochrome P450 monooxygenase in a patient.

In another embodiment the invention provides the use of a compound of formula I, or a pharmaceutically acceptable salt thereof for the manufacture of a medicament useful for increasing the blood plasma levels of a therapeutic agent which is metabolized by cytochrome P450 monooxygenase in a patient.

In another embodiment the invention provides the use of a compound of formula I, or a pharmaceutically acceptable salt thereof for the manufacture of a medicament useful for inhibiting cytochrome P450 monooxygenase in a patient.

In another embodiment the invention provides the use of a compound of formula I, or a pharmaceutically acceptable salt thereof, in combination with one or more (e.g. 1, 2, 3 or 4) therapeutic agents (e.g. agents with anti-HIV or anti-HCV properties) for the manufacture of a medicament useful for treating a viral infection (e.g., HIV, HCV) in a patient.

In another embodiment the invention provides a compound of formula I, or a pharmaceutically acceptable salt thereof, in combination with one or more (e.g. 1, 2, 3 or 4) therapeutic agents (e.g. agents with anti-HIV or anti-HCV properties) for the prophylactic or therapeutic treatment of a viral infection (e.g., HIV, HCV).

In another embodiment the invention provides processes and intermediates disclosed herein that are useful for preparing compounds of formula I or salts thereof.

### DETAILED DESCRIPTION

Reference will now be made in detail to certain claims of the invention, examples of which are illustrated in the accompanying structures and formulas. While the invention will be described in conjunction with the enumerated claims, it will be understood that they are not intended to limit the invention to those claims. On the contrary, the invention is intended to cover all alternatives, modifications, and equivalents, which may be included within the scope of the present invention as defined by the claims.

Unless otherwise indicated, all documents, patents, and patent applications referenced herein are incorporated by reference in their entirety for all purposes.

### DEFINITIONS

Unless stated otherwise, the following terms and phrases as used herein are intended to have the following meanings:

When trade names are used herein, applicants intend to independently include the tradename product and the active pharmaceutical ingredient(s) of the tradename product.

The term “alkyl” as used herein refers to a hydrocarbon containing normal, secondary or tertiary atoms. For example, an alkyl group can have 1 to 20 carbon atoms (i.e, C₁-C₂₀ alkyl), 1 to 10 carbon atoms (i.e., C₁-C₁₀ alkyl), 1 to 8 carbon atoms (i.e., C₁-C₈ alkyl) or 1 to 6 carbon atoms (i.e., C₁-C₆ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

The term “halogen” as used herein refers to fluoro, chloro, bromo and iodo.

The term “aryl” as used herein refers to a single aromatic ring or a bicyclic or multicyclic ring as described in the following definition. For example, an aryl group can have 6 to 20 carbon atoms, 6 to 14 carbon atoms, or 6 to 12 carbon atoms. Aryl includes a phenyl radical or an ortho-fused bicyclic or multicyclic radical having about 9 to 14 atoms in which at least one ring is aromatic (e.g. an aryl fused to one or more aryls or carbocycles). Such bicyclic or multicyclic rings may be optionally substituted with one or more (e.g. 1, 2 or 3) oxo groups on any carbocycle portion of the condensed ring. It is to be understood that the point of attachment of a bicyclic or multicyclic radical, as defined above, can be at any position of the ring including an aryl or a carbocycle portion of the ring. Typical aryl groups include, but are not limited to, phenyl, indenyl, naphthyl, 1,2,3,4-tetrahydronaphthyl, anthracenyl, and the like.

The term “arylalkyl” refers to an alkyl radical as defined herein in which one of the hydrogen atoms bonded to a carbon atom is replaced with an aryl radical as described herein (i.e., an aryl-alkyl- moiety). The alkyl group of the “arylalkyl” is typically 1 to 6 carbon atoms (i.e. aryl(C₁-C₆)alkyl). Arylalkyl groups include, but are not limited to, benzyl, 2-phenylethan-1-yl, 1-phenylpropan-1-yl, naphthylmethyl, 2-naphthylethan-1-yl and the like.

The term “heteroaryl” as used herein refers to a single aromatic ring or a multiple condensed ring as described in the following definition. The term “heteroaryl” includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term heteroaryl also includes multiple condensed ring systems (e.g. ring systems comprising 2 or 3 rings) wherein a heteroaryl group (as defined above) can be fused with one or more heteroaryls (e.g. naphthyridinyl), carbocycles (e.g. 5,6,7,8-tetrahydroquinolyl) or aryls (e.g. indazolyl) to form a multiple condensed ring. Such multiple condensed rings may be optionally substituted with one or more (e.g. 1, 2 or 3) oxo groups on the cycloalkyl portions of the condensed ring. It is to be understood that the point of attachment of a heteroaryl multiple condensed ring, as defined above, can be at any position of the ring including a heteroaryl, aryl or a carbocycle portion of the ring. Exemplary heteroaryls include but are not limited to pyridyl, pyrrolyl, pyrazinyl, pyrimidinyl, pyridazinyl, pyrazolyl, thienyl, indolyl, thiophenyl, imidazolyl, oxazolyl, thiazolyl, furyl, oxadiazolyl, thiadiazolyl, quinolyl, isoquinolyl, benzothiazolyl, benzoxazolyl, indazolyl, indolyl, quinoxalyl, quinazolyl, 5,6,7,8-tetrahydroisoquinolinyl and 4,5,6,7-tetrahydroindolyl.

The term “heterocyclyl” or “heterocycle” as used herein refers to a single saturated or partially unsaturated ring or a multiple condensed ring as described in the following definition. The term “heterocyclyl” or “heterocycle” includes single saturated or partially unsaturated rings (e.g. 3, 4, 5, 6, 7 or 8-membered ring) from about 1 to 7 carbon atoms and from about 1 to 3 heteroatoms selected from the group consisting of oxygen, nitrogen and sulfur in the ring. The ring may be substituted with one or more (e.g. 1, 2 or 3) oxo groups and the sulfur and nitrogen atoms may also be present in their oxidized forms. Such rings include but are not limited to azetidinyl, tetrahydrofuranyl or piperidinyl. The term heterocycle also includes multiple condensed ring systems (e.g. ring systems comprising 2 or 3 rings) wherein a heterocycle group (as defined above) can be fused with one or more heterocycles (e.g. decahydronapthyridinyl), heteroaryls (e.g. 1,2,3,4-tetrahydronaphthyridinyl), carbocycles (e.g. decahydroquinolyl) or aryls (e.g. 1,2,3,4-tetrahydroisoquinolyl) to form a multiple condensed ring. It is to be understood that the point of attachment of a heterocycle multiple condensed ring, as defined above, can be at any position of the ring including a heterocyle, heteroaryl, aryl or a carbocycle portion of the ring. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, tetrahydrothiophenyl, dihydrooxazolyl, tetrahydropyranyl, tetrahydrothiopyranyl, 1,2,3,4-tetrahydroquinolyl, benzoxazinyl and dihydrooxazolyl.

The term “heteroarylalkyl” as used herein refers to an alkyl radical as defined herein in which one of the hydrogen atoms bonded to a carbon atom is replaced with a heteroaryl radical as described herein (i.e., a heteroaryl-alkyl- moiety). The alkyl group of the “heteroarylalkyl” is typically 1 to 6 carbon atoms (i.e. heteroaryl(C₁-C₆)alkyl). Heteroarylalkyl groups include, but are not limited to heteroaryl-CH₂—, heteroaryl-CH(CH₃)—, heteroaryl-CH₂CH₂—, 2-(heteroaryl)ethan-1-yl, and the like, wherein the “heteroaryl” portion includes any of the heteroaryl groups described above. One skilled in the art will also understand that the heteroaryl group can be attached to the alkyl portion of the heteroarylalkyl by means of a carbon-carbon bond or a carbon-heteroatom bond, with the proviso that the resulting group is chemically stable. Examples of heteroarylalkyls include by way of example and not limitation 5-membered sulfur, oxygen, and/or nitrogen containing heteroaryls such as thiazolylmethyl, 2-thiazolylethan-1-yl, imidazolylmethyl, oxazolylmethyl, thiadiazolylmethyl, etc., and 6-membered sulfur, oxygen, and/or nitrogen containing heteroaryls such pyridinylmethyl, pyridizylmethyl, pyrimidylmethyl, pyrazinylmethyl, etc.

The term “heterocyclylalkyl” as used herein refers to an alkyl radical as defined herein in which one of the hydrogen atoms bonded to a carbon atom is replaced with a heterocyclyl radical as described herein (i.e., a heterocyclyl-alkyl- moiety). The alkyl group of the “heterocyclylalkyl” is typically 1 to 6 carbon atoms (i.e. heterocyclyl(C₁-C₆)alkyl). Typical heterocyclylalkyl groups include, but are not limited to heterocyclyl-CH₂—, heterocyclyl-CH(CH₃)—, heterocyclyl-CH₂CH₂—, 2-(heterocyclyl)ethan-1-yl, and the like, wherein the “heterocyclyl” portion includes any of the heterocyclyl groups described above. One skilled in the art will also understand that the heterocyclyl group can be attached to the alkyl portion of the heterocyclyl alkyl by means of a carbon-carbon bond or a carbon-heteroatom bond, with the proviso that the resulting group is chemically stable. Examples of heterocyclylalkyls include by way of example and not limitation 5-membered sulfur, oxygen, and/or nitrogen containing heterocycles such tetrahydrofuranylmethyl and pyrroldinylmethyl, etc., and 6-membered sulfur, oxygen, and/or nitrogen containing heterocycles such as piperidinylmethyl, piperazinylmethyl, morpholinylmethyl, etc.

The term “carbocycle” or “carbocyclyl” as used herein refers to a saturated (i.e., cycloalkyl) or partially unsaturated (e.g., cycloalkenyl, cycloalkadienyl, etc.) ring having 3 to 7 carbon atoms as a monocycle, 7 to 12 carbon atoms as a bicycle, and up to about 20 carbon atoms as a polycycle. Monocyclic carbocycles can also have 3 to 6 ring atoms (i.e. (C₃-C₆)carbocyclyl) as well as 5 to 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system, or spiro-fused rings. The “carbocycle” or “carbocyclyl” may be optionally substituted with one or more (e.g. 1, 2 or 3) oxo groups. Non-limiting examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

The term “carbocyclylalkyl” as used herein refers to an alkyl radical as defined herein in which one of the hydrogen atoms bonded to a carbon atom is replaced with a carbocyclyl radical as described herein (i.e., a carbocyclyl-alkyl- moiety). The alkyl group of the “carbocyclylalkyl” is typically 1 to 6 carbon atoms (i.e. carbocyclyl(C₁-C₆)alkyl). Typical carbocyclyl alkyl groups include, but are not limited to carbocyclyl-CH₂—, carbocyclyl-CH(CH₃)—, carbocyclyl-CH₂CH₂—, 2-(carbocyclyl)ethan-1-yl, and the like, wherein the “carbocyclyl” portion includes any of the carbocyclyl groups described above.

One skilled in the art will recognize that substituents and other moieties of the compounds of formula I should be selected in order to provide a compound which is sufficiently stable to provide a pharmaceutically useful compound which can be formulated into an acceptably stable pharmaceutical composition. Compounds of formula I which have such stability are contemplated as falling within the scope of the present invention.

The modifier “about” used in connection with a quantity is inclusive of the stated value and has the meaning dictated by the context (e.g., includes the degree of error associated with measurement of the particular quantity).

### Protecting Groups

Protecting groups are available, commonly known and used, and are optionally used to prevent side reactions with the protected group during synthetic procedures, i.e. routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. The PG groups do not need to be, and generally are not, the same if the compound is substituted with multiple PG groups. In general, PG groups will be used to protect functional groups such as carboxyl, hydroxyl, thio, or amino groups and to thus prevent side reactions or to otherwise facilitate the synthetic efficiency. The order of deprotection to yield free, deprotected groups is dependent upon the intended direction of the synthesis and the reaction conditions to be encountered, and may occur in any order as determined by the artisan.

Various functional groups of the compounds of the invention may be protected. For example, protecting groups for —OH groups (whether hydroxyl, carboxylic acid, phosphonic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in Protective Groups in Organic Synthesis, Theodora W. Greene and Peter G. M. Wuts (John Wiley & Sons, Inc., New York, 1999, ISBN 0-471-16019-9) (“Greene”). See also Kocienski, Philip J.; Protecting Groups (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phosphonic acid, phosphonate, sulfonic acid and other protecting groups for acids see Greene as set forth below. Such groups include by way of example and not limitation, esters, amides, hydrazides, and the like. Ester-forming groups include: (1) phosphonate ester-forming groups, such as phosphonamidate esters, phosphorothioate esters, phosphonate esters, and phosphon-bis-amidates; (2) carboxyl ester-forming groups, and (3) sulphur ester-forming groups, such as sulphonate, sulfate, and sulfinate.

### Stereoisomers

The term “stereoisomers” refers to compounds which have identical chemical constitution, but differ with regard to the arrangement of the atoms or groups in space (e.g. diastereomers and enantiomers).

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

One skilled in the art will recognize that stereoisomers or mixtures of stereoisomers of the compounds of the invention include enantiomers, diastereomers, and other stereoisomers. For example, for a compound of formula I with the following structure:

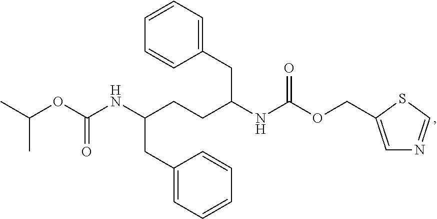 [SMILES: CC(C)OC(=O)NC(CCC(Cc1ccccc1)NC(=O)OCc1cncs1)Cc1ccccc1]

contemplated stereoisomers include at least:

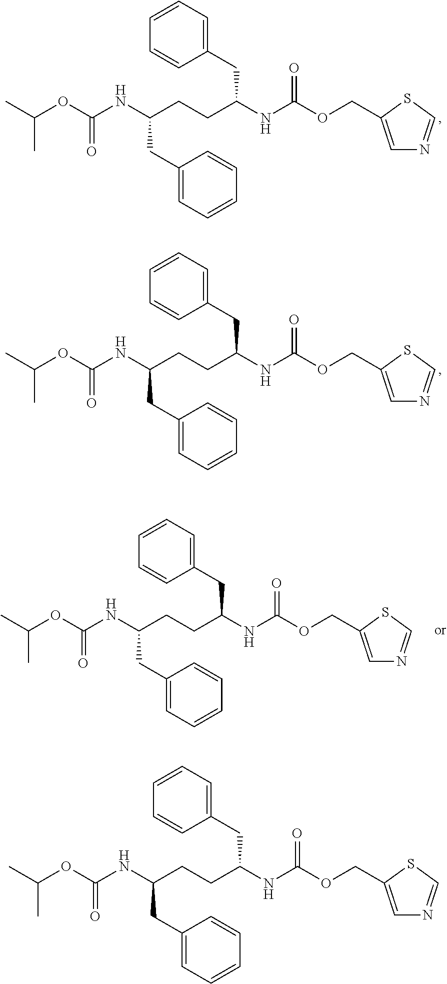

as well as mixtures of two or more of these stereoisomers. Thus, it is to be understood that when a bond is drawn in a non-stereochemical manner (e.g. flat) for a compound of the invention, the atom to which the bond is attached includes all stereochemical possibilities.

It is also to understood that when a bond is drawn in a defined stereochemical manner (e.g. bold, bold-wedge, dashed or dashed-wedge), the atom to which the stereochemical bond is attached has the stereochemistry as shown unless otherwise noted. Thus, for a compound of the following formula:

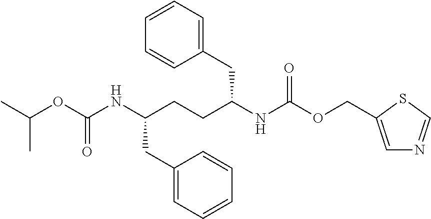 [SMILES: CC(C)OC(=O)N[C@H](CC[C@H](Cc1ccccc1)NC(=O)OCc1cncs1)Cc1ccccc1]

the stereochemistry of the compound of the formula is as shown.

### Compounds of Formula I

In one embodiment of the compounds of formula I, X is R^{1a}OC(O)—.

In another embodiment of the compounds of formula I, X is R^{1b}C(O)—.

In another embodiment of the compounds of formula I, X is R^{1b}S(O)₂—.

In another embodiment of the compounds of formula I, X is R^{1a}(R^{a})NC(O)—.

In another embodiment of the compounds of formula I, X is R^{1a}(R^{a})NS(O)₂—.

In another embodiment of the compounds of formula I, X is selected from the group consisting of R^{1a}OC(O)—, R^{1b}C(O)—, R^{1a}(R^{a})NC(O)— and R^{1b}S(O)₂—.

In one embodiment of the compounds of formula I, R^{a} is H.

In another embodiment of the compounds of formula I, R^{a} is (C₁-C₆)alkyl.

In one embodiment of the compounds of formula I, R^{1a} is (C₁-C₈)alkyl, (C₃-C₆)carbocyclyl or aryl(C₁-C₆)alkyl, wherein any (C₁-C₈)alkyl of R^{1a} is optionally substituted with one or more Z³ groups and wherein any (C₃-C₆)carbocyclyl or aryl(C₁-C₆)alkyl of R^{1a} is optionally substituted with one or more Z⁴ groups.

In another embodiment of the compounds of formula I, R^{1a} is propyl, butyl, pentyl, 2-methylpentyl, 2,5-dimethylhexyl, 2,2-dimethylpentyl, phenylpropyl, pentylcyclohexyl, (prop-2-yl)cyclohexyl, methylcyclohexyl or (4-chlorophenyl)cyclohexyl.

In another embodiment of the compounds of formula I, R^{1a} is propyl, butyl, pentyl, 2-methylpentyl, 2,5-dimethylhexyl, 2,2-dimethylpentyl, phenylpropyl, 4-pentylcyclohexyl, 4-(prop-2-yl)cyclohexyl, 4-methylcyclohexyl or 4-(4-chlorophenyl)cyclohexyl.

In another embodiment of the compounds of formula I, R^{1a} is prop-2-yl, but-2-yl, pent-3-yl, 4-methylpent-2-yl, 2,5-dimethylhex-3-yl, 4,4-dimethylpent-2-yl, 1-phenylprop-1-yl, 4-pentylcyclohexyl, 4-(prop-2-yl)cyclohexyl, 4-methylcyclohexyl or 4-(4-chlorophenyl)cyclohexyl.

In one embodiment of the compounds of formula I, R^{1b} is (C₁-C₈)alkyl, (C₃-C₆)carbocyclyl or aryl(C₁-C₆)alkyl, wherein any (C₁-C₈)alkyl of R^{1b} is optionally substituted with one or more Z³ groups and wherein any (C₃-C₆)carbocyclyl or aryl(C₁-C₆)alkyl of R^{1b} is optionally substituted with one or more Z⁴ groups.

In another embodiment of the compounds of formula I, R^{1b} is propyl, butyl, pentyl, 2-methylpentyl, 2,5-dimethylhexyl, 2,2-dimethylpentyl, phenylpropyl, pentylcyclohexyl, (prop-2-yl)cyclohexyl, methylcyclohexyl or (4-chlorophenyl)cyclohexyl.

In another embodiment of the compounds of formula I, R^{1b} is propyl, butyl, pentyl, 2-methylpentyl, 2,5-dimethylhexyl, 2,2-dimethylpentyl, phenylpropyl, 4-pentylcyclohexyl, 4-(prop-2-yl)cyclohexyl, 4-methylcyclohexyl or 4-(4-chlorophenyl)cyclohexyl.

In another embodiment of the compounds of formula I, R^{1b} is prop-2-yl, but-2-yl, pent-3-yl, 4-methylpent-2-yl, 2,5-dimethylhex-3-yl, 4,4-dimethylpent-2-yl, 1-phenylprop-1-yl, 4-pentylcyclohexyl, 4-(prop-2-yl)cyclohexyl, 4-methylcyclohexyl or 4-(4-chlorophenyl)cyclohexyl.

In one embodiment of the compounds of formula I, each Z⁴ group is independently selected from (C₁-C₆)alkyl and aryl, wherein any aryl of Z⁴ is optionally substituted with one or more halogen.

In another embodiment of the compounds of formula I, each Z⁴ group is independently selected from methyl, pentyl, isopropyl and 4-chlorophenyl.

In one embodiment of the compounds of formula I, A¹ is aryl(C₁-C₆)alkyl, wherein any aryl(C₁-C₆)alkyl of A¹ is optionally substituted with one or more Z² groups.

In another embodiment of the compounds of formula I, A¹ is aryl-CH₂—, wherein any aryl-CH₂— of A¹ is optionally substituted with one or more Z² groups.

In another embodiment of the compounds of formula I, A¹ is phenyl(C₁-C₆)alkyl, wherein any phenyl(C₁-C₆)alkyl of A¹ is optionally substituted with one or more Z² groups.

In another embodiment of the compounds of formula I, A¹ is phenyl-CH₂—, wherein any phenyl-CH₂— of A¹ is optionally substituted with one or more Z² groups.

In another embodiment of the compounds of formula I, A¹ is aryl-CH₂—.

In another embodiment of the compounds of formula I, A¹ is phenyl-CH₂—.

In one embodiment of the compounds of formula I, A² is aryl(C₁-C₆)alkyl, wherein any aryl(C₁-C₆)alkyl of A² is optionally substituted with one or more Z² groups.

In another embodiment of the compounds of formula I, A² is aryl-CH₂—, wherein any aryl-CH₂— of A² is optionally substituted with one or more Z² groups.

In another embodiment of the compounds of formula I, A² is phenyl(C₁-C₆)alkyl, wherein any phenyl(C₁-C₆)alkyl of A² is optionally substituted with one or more Z² groups.

In another embodiment of the compounds of formula I, A² is phenyl-CH₂—, wherein any phenyl-CH₂— of A² is optionally substituted with one or more Z² groups.

In another embodiment of the compounds of formula I, A² is aryl-CH₂—.

In another embodiment of the compounds of formula I, A² is phenyl-CH₂—.

In one embodiment of the compounds of formula I, Y is —C(O)O—.

In another embodiment of the compounds of formula I, Y is —C(O)N(R^{b})—.

In one embodiment of the compounds of formula I, R^{b} is H or (C₁-C₆)alkyl.

In another embodiment of the compounds of formula I, R^{b} is H.

In another embodiment of the compounds of formula I, R^{b} is (C₁-C₆)alkyl.

In one embodiment of the compounds of formula I, R⁴ is heteroaryl(C₁-C₆)alkyl, wherein the heteroaryl(C₁-C₆)alkyl of R⁴ is optionally substituted with one or more Z⁵.

In another embodiment of the compounds of formula I, R⁴ is thiazolyl(C₁-C₆)alkyl, wherein thiazolyl is optionally substituted with one or more Z⁵ groups.

In another embodiment of the compounds of formula I, R⁴ is heteroaryl(C₁-C₆)alkyl.

In another embodiment of the compounds of formula I, R⁴ is heteroarylmethyl.

In another embodiment of the compounds of formula I, R⁴ is thiazolylmethyl.

In another embodiment of the compounds of formula I, R⁴ is thiazol-5-ylmethyl.

In one embodiment of the compounds of formula I, R² is H or (C₁-C₆)alkyl.

In another embodiment of the compounds of formula I, R² is H.

In another embodiment of the compounds of formula I, R² is (C₁-C₆)alkyl.

In one embodiment of the compounds of formula I, R³ is H or (C₁-C₆)alkyl.

In another embodiment of the compounds of formula I, R³ is H.

In another embodiment of the compounds of formula I, R³ is (C₁-C₆)alkyl.

In one embodiment the invention provides a compound of formula I selected from:

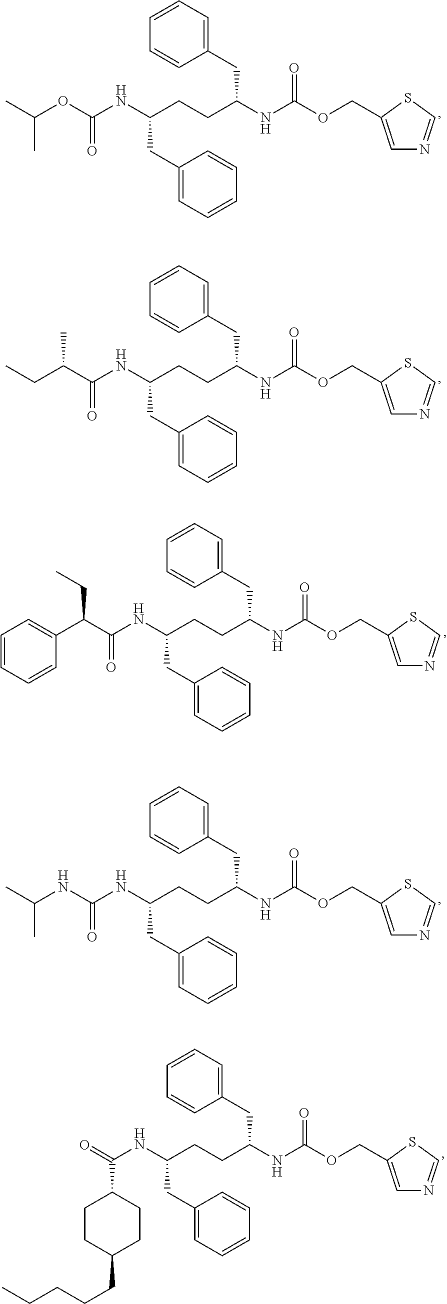

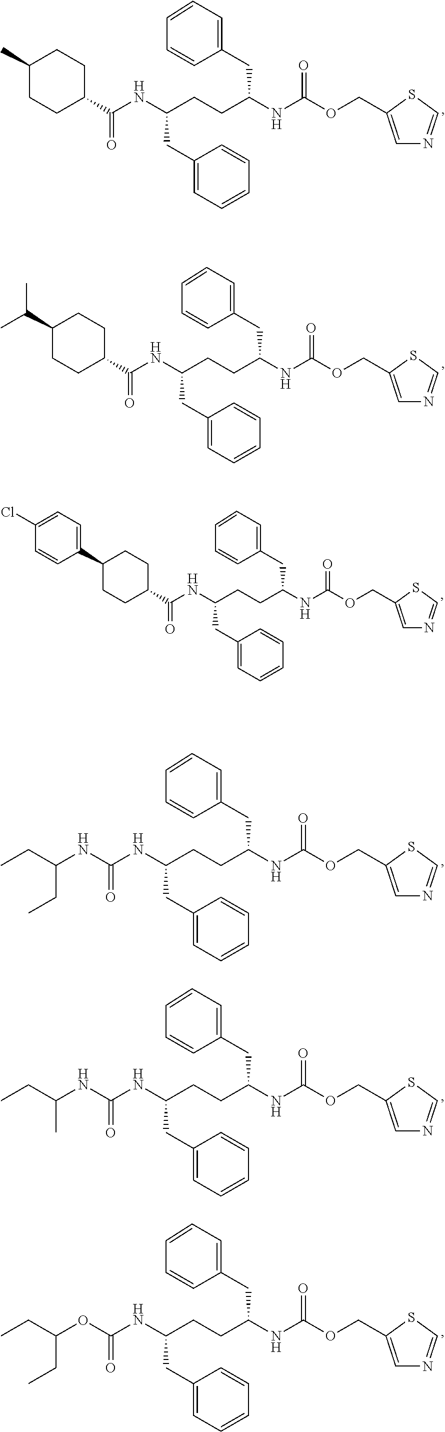

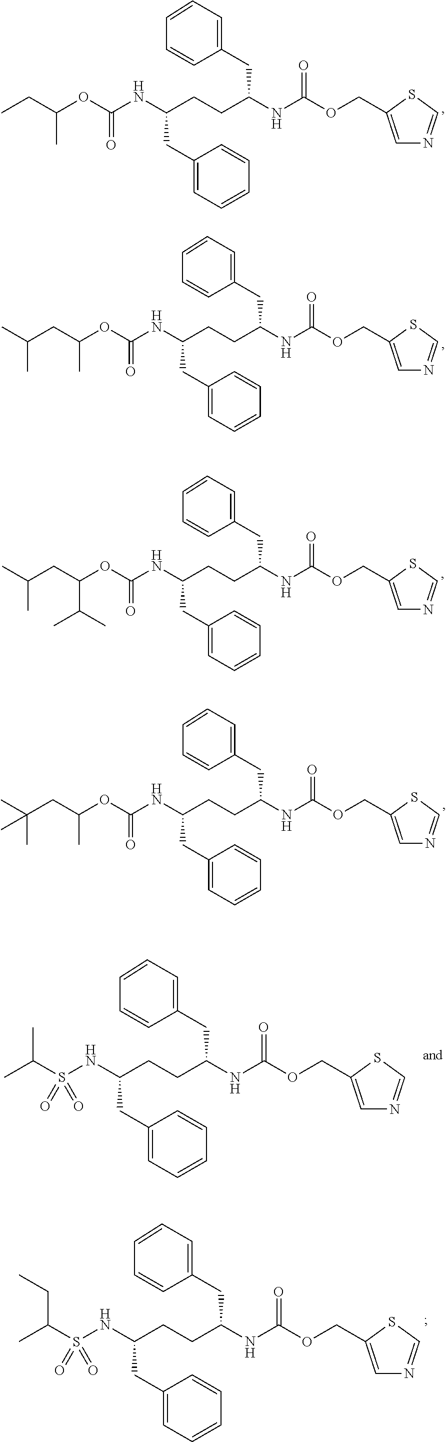

and salts thereof.

In one embodiment, the compound of formula I, has the structure of formula Ia:

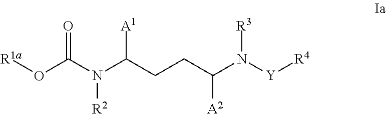

or a salt thereof, wherein A¹, A², R^{1a}, R², R³, R⁴, and Y are as defined herein.

In one embodiment of the compounds of formula Ia, R^{1a} is (C₁-C₈)alkyl, (C₃-C₆)carbocyclyl or aryl(C₁-C₆)alkyl, wherein any (C₁-C₈)alkyl of R^{1a} is optionally substituted with one or more Z³ groups and wherein any (C₃-C₆)carbocyclyl or aryl(C₁-C₆)alkyl of R^{1a} is optionally substituted with one or more Z⁴ groups.

In another embodiment of the compounds of formula Ia, R^{1a} is (C₁-C₈)alkyl, wherein any (C₁-C₈)alkyl of R^{1a} is optionally substituted with one or more Z³ groups.

In another embodiment of the compounds of formula Ia, R^{1a} is propyl, butyl, pentyl, 2-methylpentyl, 2,5-dimethylhexyl or 2,2-dimethylpentyl,

In another embodiment of the compounds of formula Ia, R^{1a} is propyl, butyl, pentyl, 2-methylpentyl, 2,5-dimethylhexyl, 2,2-dimethylpentyl, phenylpropyl, pentylcyclohexyl, (prop-2-yl)cyclohexyl, methylcyclohexyl or (4-chlorophenyl)cyclohexyl.

In another embodiment of the compounds of formula Ia, R^{1a} is propyl, butyl, pentyl, 2-methylpentyl, 2,5-dimethylhexyl, 2,2-dimethylpentyl, phenylpropyl, 4-pentylcyclohexyl, 4-(prop-2-yl)cyclohexyl, 4-methylcyclohexyl or 4-(4-chlorophenyl)cyclohexyl.

In another embodiment of the compounds of formula Ia, R^{1a} is prop-2-yl, but-2-yl, pent-3-yl, 4-methylpent-2-yl, 2,5-dimethylhex-3-yl or 4,4-dimethylpent-2-yl.

In another embodiment of the compounds of formula Ia, R^{1a} is prop-2-yl, but-2-yl, pent-3-yl, 4-methylpent-2-yl, 2,5-dimethylhex-3-yl, 4,4-dimethylpent-2-yl, 1-phenylprop-1-yl, 4-pentylcyclohexyl, 4-(prop-2-yl)cyclohexyl, 4-methylcyclohexyl or 4-(4-chlorophenyl)cyclohexyl.

In one embodiment of the compounds of formula Ia, each Z⁴ group is independently selected from (C₁-C₆)alkyl and aryl, wherein any aryl of Z⁴ is optionally substituted with one or more halogen.

In one embodiment of the compounds of formula Ia, A¹ is aryl(C₁-C₆)alkyl, wherein any aryl(C₁-C₆)alkyl of A¹ is optionally substituted with one or more Z² groups.

In another embodiment of the compounds of formula Ia, A¹ is aryl-CH₂—, wherein any aryl-CH₂— of A¹ is optionally substituted with one or more Z² groups.

In another embodiment of the compounds of formula Ia, A¹ is phenyl(C₁-C₆)alkyl, wherein any phenyl(C₁-C₆)alkyl of A¹ is optionally substituted with one or more Z² groups.

In another embodiment of the compounds of formula Ia, A¹ is phenyl-CH₂—, wherein any phenyl-CH₂— of A¹ is optionally substituted with one or more Z² groups.

In another embodiment of the compounds of formula Ia, A¹ is aryl-CH₂—.

In another embodiment of the compounds of formula Ia, A¹ is phenyl-CH₂—.

In one embodiment of the compounds of formula Ia, A² is aryl(C₁-C₆)alkyl, wherein any aryl(C₁-C₆)alkyl of A² is optionally substituted with one or more Z² groups.

In another embodiment of the compounds of formula Ia, A² is aryl-CH₂—, wherein any aryl-CH₂— of A² is optionally substituted with one or more Z² groups.

In another embodiment of the compounds of formula Ia, A² is phenyl(C₁-C₆)alkyl, wherein any phenyl(C₁-C₆)alkyl of A² is optionally substituted with one or more Z² groups.

In another embodiment of the compounds of formula Ia, A² is phenyl-CH₂—, wherein any phenyl-CH₂— of A² is optionally substituted with one or more Z² groups.

In another embodiment of the compounds of formula Ia, A² is aryl-CH₂—.

In another embodiment of the compounds of formula Ia, A² is phenyl-CH₂—.

In one embodiment of the compounds of formula Ia, Y is —C(O)O—.

In another embodiment of the compounds of formula Ia, Y is —C(O)N(R^{b})—.

In one embodiment of the compounds of formula Ia, R^{b} is H or (C₁-C₆)alkyl.

In another embodiment of the compounds of formula Ia, R^{b} is H.

In another embodiment of the compounds of formula Ia, R^{b} is (C₁-C₆)alkyl.

In one embodiment of the compounds of formula Ia, R⁴ is heteroaryl(C₁-C₆)alkyl,

wherein the heteroaryl(C₁-C₆)alkyl of R⁴ is optionally substituted with one or more Z⁵ groups.

In another embodiment of the compounds of formula Ia, R⁴ is thiazolyl(C₁-C₆)alkyl, wherein thiazolyl is optionally substituted with one or more Z⁵ groups.

In another embodiment of the compounds of formula Ia, R⁴ is heteroaryl(C₁-C₆)alkyl.

In another embodiment of the compounds of formula Ia, R⁴ is heteroarylmethyl.

In another embodiment of the compounds of formula Ia, R⁴ is thiazolylmethyl.

In another embodiment of the compounds of formula Ia, R⁴ is thiazol-5-ylmethyl.

In one embodiment of the compounds of formula Ia, R² is H or (C₁-C₆)alkyl.

In another embodiment of the compounds of formula Ia, R² is H.

In another embodiment of the compounds of formula Ia, R² is (C₁-C₆)alkyl.

In one embodiment of the compounds of formula Ia, R³ is H or (C₁-C₆)alkyl.

In another embodiment of the compounds of formula Ia, R³ is H.

In another embodiment of the compounds of formula Ia, R³ is (C₁-C₆)alkyl.

In one embodiment, the compound of formula I, has the structure of formula Ib:

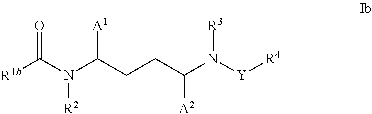

or a salt thereof, wherein A¹, A², R^{1b}, R², R³, R⁴, and Y are as defined herein.

In one embodiment of the compounds of formula Ib, R^{1b} is (C₁-C₈)alkyl, (C₃-C₆)carbocyclyl or aryl(C₁-C₆)alkyl, wherein any (C₁-C₈)alkyl of R^{1b} is optionally substituted with one or more Z³ groups and wherein any (C₃-C₆)carbocyclyl or aryl(C₁-C₆)alkyl of R^{1b} is optionally substituted with one or more Z⁴ groups.

In another embodiment of the compounds of formula Ib, R^{1b} is propyl, butyl, pentyl, 2-methylpentyl, 2,5-dimethylhexyl, 2,2-dimethylpentyl, phenylpropyl, pentylcyclohexyl, (prop-2-yl)cyclohexyl, methylcyclohexyl or (4-chlorophenyl)cyclohexyl.

In another embodiment of the compounds of formula Ib, R^{1b} is butyl, phenylpropyl, pentylcyclohexyl, (prop-2-yl)cyclohexyl, methylcyclohexyl or (4-chlorophenyl)cyclohexyl.

In another embodiment of the compounds of formula Ib, R^{1b} is propyl, butyl, pentyl, 2-methylpentyl, 2,5-dimethylhexyl, 2,2-dimethylpentyl, or phenylpropyl, 4-pentylcyclohexyl, 4-(prop-2-yl)cyclohexyl, 4-methylcyclohexyl or 4-(4-chlorophenyl)cyclohexyl.

In another embodiment of the compounds of formula Ib, R^{1b} is butyl, phenylpropyl, 4-pentylcyclohexyl, 4-(prop-2-yl)cyclohexyl, 4-methylcyclohexyl or 4-(4-chlorophenyl)cyclohexyl.

In another embodiment of the compounds of formula Ib, R^{1b} is prop-2-yl, but-2-yl, pent-3-yl, 4-methylpent-2-yl, 2,5-dimethylhex-3-yl, 4,4-dimethylpent-2-yl, 1-phenylprop-1-yl, 4-pentylcyclohexyl, 4-(prop-2-yl)cyclohexyl, 4-methylcyclohexyl or 4-(4-chlorophenyl)cyclohexyl.

In another embodiment of the compounds of formula Ib, R^{1b} is but-2-yl, 1-phenylprop-1-yl, 4-pentylcyclohexyl, 4-(prop-2-yl)cyclohexyl, 4-methylcyclohexyl or 4-(4-chlorophenyl)cyclohexyl.

In one embodiment of the compounds of formula Ib, each Z⁴ group is independently selected from (C₁-C₆)alkyl and aryl, wherein any aryl of Z⁴ is optionally substituted with one or more halogen.

In another embodiment of the compounds of formula Ib, each Z⁴ group is independently selected from methyl, pentyl, isopropyl and 4-chlorophenyl.

In one embodiment of the compounds of formula Ib, A¹ is aryl(C₁-C₆)alkyl, wherein any aryl(C₁-C₆)alkyl of A¹ is optionally substituted with one or more Z² groups.

In another embodiment of the compounds of formula Ib, A¹ is aryl-CH₂—, wherein any aryl-CH₂— of A¹ is optionally substituted with one or more Z² groups.

In another embodiment of the compounds of formula Ib, A¹ is phenyl(C₁-C₆)alkyl, wherein any phenyl(C₁-C₆)alkyl of A¹ is optionally substituted with one or more Z² groups.

In another embodiment of the compounds of formula Ib, A¹ is phenyl-CH₂—, wherein any phenyl-CH₂— of A¹ is optionally substituted with one or more Z² groups.

In another embodiment of the compounds of formula Ib, A¹ is aryl-CH₂—.

In another embodiment of the compounds of formula Ib, A¹ is phenyl-CH₂—.

In one embodiment of the compounds of formula Ib, A² is aryl(C₁-C₆)alkyl, wherein any aryl(C₁-C₆)alkyl of A² is optionally substituted with one or more Z² groups.

In another embodiment of the compounds of formula Ib, A² is aryl-CH₂—, wherein any aryl-CH₂— of A² is optionally substituted with one or more Z² groups.

In another embodiment of the compounds of formula Ib, A² is phenyl(C₁-C₆)alkyl, wherein any phenyl(C₁-C₆)alkyl of A² is optionally substituted with one or more Z² groups.

In another embodiment of the compounds of formula Ib, A² is phenyl-CH₂—, wherein any phenyl-CH₂— of A² is optionally substituted with one or more Z² groups.

In another embodiment of the compounds of formula Ib, A² is aryl-CH₂—.

In another embodiment of the compounds of formula Ib, A² is phenyl-CH₂—.

In one embodiment of the compounds of formula Ib, Y is —C(O)O—.

In another embodiment of the compounds of formula Ib, Y is —C(O)N(R^{b})—.

In one embodiment of the compounds of formula Ib, R^{b} is H or (C₁-C₆)alkyl.

In another embodiment of the compounds of formula Ib, R^{b} is H.

In another embodiment of the compounds of formula Ib, R^{b} is (C₁-C₆)alkyl.

In one embodiment of the compounds of formula Ib, R⁴ is heteroaryl(C₁-C₆)alkyl, wherein the heteroaryl(C₁-C₆)alkyl of R⁴ is optionally substituted with one or more Z⁵ groups.

In another embodiment of the compounds of formula Ib, R⁴ is thiazolyl(C₁-C₆)alkyl, wherein thiazolyl is optionally substituted with one or more Z⁵ groups.

In another embodiment of the compounds of formula Ib, R⁴ is heteroaryl(C₁-C₆)alkyl.

In another embodiment of the compounds of formula Ib, R⁴ is heteroarylmethyl.

In another embodiment of the compounds of formula Ib, R⁴ is thiazolylmethyl.

In another embodiment of the compounds of formula Ib, R⁴ is thiazol-5-ylmethyl.

In one embodiment of the compounds of formula Ib, R² is H or (C₁-C₆)alkyl.

In another embodiment of the compounds of formula Ib, R² is H.

In another embodiment of the compounds of formula Ib, R² is (C₁-C₆)alkyl.

In one embodiment of the compounds of formula Ib, R³ is H or (C₁-C₆)alkyl.

In another embodiment of the compounds of formula Ib, R³ is H.

In another embodiment of the compounds of formula Ib, R³ is (C₁-C₆)alkyl.

In one embodiment, the compound of formula I, has the structure of formula Ic:

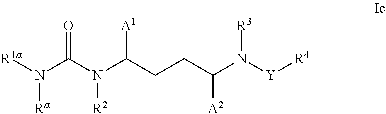

or a salt thereof, wherein A¹, A², R^{1a}, R², R³, R⁴, R^{a} and Y are as defined herein.

In one embodiment of the compounds of formula Ic, R^{a} is H.

In another embodiment of the compounds of formula Ic, R^{a} is (C₁-C₆)alkyl.

In one embodiment of the compounds of formula Ic, R^{1a} is (C₁-C₈)alkyl, (C₃-C₆)carbocyclyl or aryl(C₁-C₆)alkyl, wherein any (C₁-C₈)alkyl of R^{1a} is optionally substituted with one or more Z³ groups and wherein any (C₃-C₆)carbocyclyl or aryl(C₁-C₆)alkyl of R^{1a} is optionally substituted with one or more Z⁴ groups.

In another embodiment of the compounds of formula Ic, R^{1a} is (C₁-C₈)alkyl, wherein any (C₁-C₈)alkyl of R^{1a} is optionally substituted with one or more Z³ groups.

In another embodiment of the compounds of formula Ic, R^{1a} is propyl, butyl, pentyl, 2-methylpentyl, 2,5-dimethylhexyl, 2,2-dimethylpentyl, phenylpropyl, pentylcyclohexyl, (prop-2-yl)cyclohexyl, methylcyclohexyl or (4-chlorophenyl)cyclohexyl.

In another embodiment of the compounds of formula Ic, R^{1a} is propyl, butyl, or pentyl.

In another embodiment of the compounds of formula Ic, R^{1a} is propyl, butyl, pentyl, 2-methylpentyl, 2,5-dimethylhexyl, 2,2-dimethylpentyl, phenylpropyl, 4-pentylcyclohexyl, 4-(prop-2-yl)cyclohexyl, 4-methylcyclohexyl or 4-(4-chlorophenyl)cyclohexyl.

In another embodiment of the compounds of formula Ic, R^{1a} is prop-2-yl, but-2-yl, pent-3-yl, 4-methylpent-2-yl, 2,5-dimethylhex-3-yl, 4,4-dimethylpent-2-yl, 1-phenylprop-1-yl, 4-pentylcyclohexyl, 4-(prop-2-yl)cyclohexyl, 4-methylcyclohexyl or 4-(4-chlorophenyl)cyclohexyl.

In another embodiment of the compounds of formula Ic, R^{1a} is prop-2-yl, but-2-yl or pent-3-yl.

In one embodiment of the compounds of formula Ic, each Z⁴ group is independently selected from (C₁-C₆)alkyl and aryl, wherein any aryl of Z⁴ is optionally substituted with one or more halogen.

In one embodiment of the compounds of formula Ic, A¹ is aryl(C₁-C₆)alkyl, wherein any aryl(C₁-C₆)alkyl of A¹ is optionally substituted with one or more Z² groups.

In another embodiment of the compounds of formula Ic, A¹ is aryl-CH₂—, wherein any aryl-CH₂— of A¹ is optionally substituted with one or more Z² groups.

In another embodiment of the compounds of formula Ic, A¹ is phenyl(C₁-C₆)alkyl, wherein any phenyl(C₁-C₆)alkyl of A¹ is optionally substituted with one or more Z² groups.

In another embodiment of the compounds of formula Ic, A¹ is phenyl-CH₂—, wherein any phenyl-CH₂— of A¹ is optionally substituted with one or more Z² groups.

In another embodiment of the compounds of formula Ic, A¹ is aryl-CH₂—.

In another embodiment of the compounds of formula Ic, A¹ is phenyl-CH₂—.

In one embodiment of the compounds of formula Ic, A² is aryl(C₁-C₆)alkyl, wherein any aryl(C₁-C₆)alkyl of A² is optionally substituted with one or more Z² groups.

In another embodiment of the compounds of formula Ic, A² is aryl-CH₂—, wherein any aryl-CH₂— of A² is optionally substituted with one or more Z² groups.

In another embodiment of the compounds of formula Ic, A² is phenyl(C₁-C₆)alkyl, wherein any phenyl(C₁-C₆)alkyl of A² is optionally substituted with one or more Z² groups.

In another embodiment of the compounds of formula Ic, A² is phenyl-CH₂—, wherein any phenyl-CH₂— of A² is optionally substituted with one or more Z² groups.

In another embodiment of the compounds of formula Ic, A² is aryl-CH₂—.

In another embodiment of the compounds of formula Ic, A² is phenyl-CH₂—.

In one embodiment of the compounds of formula Ic, Y is —C(O)O—.

In another embodiment of the compounds of formula Ic, Y is —C(O)N(R^{b})—.

In one embodiment of the compounds of formula Ic, R^{b} is H or (C₁-C₆)alkyl.

In another embodiment of the compounds of formula Ic, R^{b} is H.

In another embodiment of the compounds of formula Ic, R^{b} is (C₁-C₆)alkyl.

In one embodiment of the compounds of formula Ic, R⁴ is heteroaryl(C₁-C₆)alkyl, wherein the heteroaryl(C₁-C₆)alkyl of R⁴ is optionally substituted with one or more Z⁵ groups.

In another embodiment of the compounds of formula Ic, R⁴ is thiazolyl(C₁-C₆)alkyl, wherein thiazolyl is optionally substituted with one or more Z⁵ groups.

In another embodiment of the compounds of formula Ic, R⁴ is heteroaryl(C₁-C₆)alkyl.

In another embodiment of the compounds of formula Ic, R⁴ is heteroarylmethyl.

In another embodiment of the compounds of formula Ic, R⁴ is thiazolylmethyl.

In another embodiment of the compounds of formula Ic, R⁴ is thiazol-5-ylmethyl.

In one embodiment of the compounds of formula Ic, R² is H or (C₁-C₆)alkyl.

In another embodiment of the compounds of formula Ic, R² is H.

In another embodiment of the compounds of formula Ic, R² is (C₁-C₆)alkyl.

In one embodiment of the compounds of formula Ic, R³ is H or (C₁-C₆)alkyl.

In another embodiment of the compounds of formula Ic, R³ is H.

In another embodiment of the compounds of formula Ic, R³ is (C₁-C₆)alkyl.

In one embodiment, the compound of formula I, has the structure of formula Id:

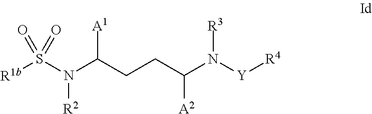

or a salt thereof, wherein A¹, A², R^{1b}, R², R³, R⁴, and Y are as defined herein.

In one embodiment of the compounds of formula Id, R^{1b} is (C₁-C₈)alkyl, (C₃-C₆)carbocyclyl or aryl(C₁-C₆)alkyl, wherein any (C₁-C₈)alkyl of R^{1b} is optionally substituted with one or more Z³ groups, and wherein any (C₃-C₆)carbocyclyl or aryl(C₁-C₆)alkyl of R^{1b} is optionally substituted with one or more Z⁴ groups.

In another embodiment of the compounds of formula Id, R^{1b} is (C₁-C₈)alkyl, wherein any (C₁-C₈)alkyl of R^{1b} is optionally substituted with one or more Z³ groups.

In another embodiment of the compounds of formula Id, R^{1b} is propyl, butyl, pentyl, 2-methylpentyl, 2,5-dimethylhexyl, 2,2-dimethylpentyl, phenylpropyl, pentylcyclohexyl, (prop-2-yl)cyclohexyl, methylcyclohexyl or (4-chlorophenyl)cyclohexyl.

In another embodiment of the compounds of formula Id, R^{1b} is propyl or butyl.

In another embodiment of the compounds of formula Id, R^{1b} is propyl, butyl, pentyl, 2-methylpentyl, 2,5-dimethylhexyl, 2,2-dimethylpentyl, phenylpropyl, 4-pentylcyclohexyl, 4-(prop-2-yl)cyclohexyl, 4-methylcyclohexyl or 4-(4-chlorophenyl)cyclohexyl.

In another embodiment of the compounds of formula Id, R^{1b} is prop-2-yl, but-2-yl, pent-3-yl, 4-methylpent-2-yl, 2,5-dimethylhex-3-yl, 4,4-dimethylpent-2-yl, 1-phenylprop-1-yl, 4-pentylcyclohexyl, 4-(prop-2-yl)cyclohexyl, 4-methylcyclohexyl or 4-(4-chlorophenyl)cyclohexyl.

In another embodiment of the compounds of formula Id, R^{1b} is prop-2-yl or but-2-yl.

In one embodiment of the compounds of formula Id, each Z⁴ group is independently selected from (C₁-C₆)alkyl and aryl, wherein any aryl of Z⁴ is optionally substituted with one or more halogen.

In one embodiment of the compounds of formula Id, A¹ is aryl(C₁-C₆)alkyl, wherein any aryl(C₁-C₆)alkyl of A¹ is optionally substituted with one or more Z² groups.

In another embodiment of the compounds of formula Id, A¹ is aryl-CH₂—, wherein any aryl-CH₂— of A¹ is optionally substituted with one or more Z² groups.

In another embodiment of the compounds of formula Id, A¹ is phenyl(C₁-C₆)alkyl, wherein any phenyl(C₁-C₆)alkyl of A¹ is optionally substituted with one or more Z² groups.

In another embodiment of the compounds of formula Id, A¹ is phenyl-CH₂—, wherein any phenyl-CH₂— of A¹ is optionally substituted with one or more Z² groups.

In another embodiment of the compounds of formula Id, A¹ is aryl-CH₂—.

In another embodiment of the compounds of formula Id, A¹ is phenyl-CH₂—.

In one embodiment of the compounds of formula Id, A² is aryl(C₁-C₆)alkyl, wherein any aryl(C₁-C₆)alkyl of A² is optionally substituted with one or more Z² groups.

In another embodiment of the compounds of formula Id, A² is aryl-CH₂—, wherein any aryl-CH₂— of A² is optionally substituted with one or more Z² groups.

In another embodiment of the compounds of formula Id, A² is phenyl(C₁-C₆)alkyl, wherein any phenyl(C₁-C₆)alkyl of A² is optionally substituted with one or more Z² groups.

In another embodiment of the compounds of formula I, A² is phenyl-CH₂—, wherein any phenyl-CH₂— of A² is optionally substituted with one or more Z² groups.

In another embodiment of the compounds of formula I, A² is aryl-CH₂—.

In another embodiment of the compounds of formula I, A² is phenyl-CH₂—.

In one embodiment of the compounds of formula Id, Y is —C(O)O—.

In another embodiment of the compounds of formula Id, Y is —C(O)N(R^{b})—.

In one embodiment of the compounds of formula Id, R^{b} is H or (C₁-C₆)alkyl.

In another embodiment of the compounds of formula Id, R^{b} is H.

In another embodiment of the compounds of formula Id, R^{b} is (C₁-C₆)alkyl.

In one embodiment of the compounds of formula Id, R⁴ is heteroaryl(C₁-C₆)alkyl, wherein the heteroaryl(C₁-C₆)alkyl of R⁴ is optionally substituted with one or more Z⁵ groups.

In another embodiment of the compounds of formula Id, R⁴ is thiazolyl(C₁-C₆)alkyl, wherein thiazolyl is optionally substituted with one or more Z⁵ groups.

In another embodiment of the compounds of formula Id, R⁴ is heteroaryl(C₁-C₆)alkyl.

In another embodiment of the compounds of formula Id, R⁴ is heteroarylmethyl.

In another embodiment of the compounds of formula Id, R⁴ is thiazolylmethyl.

In another embodiment of the compounds of formula Id, R⁴ is thiazol-5-ylmethyl.

In one embodiment of the compounds of formula Id, R² is H or (C₁-C₆)alkyl.

In another embodiment of the compounds of formula Id, R² is H.

In another embodiment of the compounds of formula Id, R² is (C₁-C₆)alkyl.

In one embodiment of the compounds of formula Id, R³ is H or (C₁-C₆)alkyl.

In another embodiment of the compounds of formula Id, R³ is H.

In another embodiment of the compounds of formula Id, R³ is (C₁-C₆)alkyl.

In one embodiment, the compound of formula I, has the structure of formula Ie:

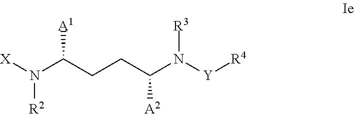

or a salt thereof, wherein A¹, A², R², R³, R⁴, X and Y are as defined herein.

In one embodiment, the compounds of formula Ie are at least 60% a single stereoisomer at both the carbon attached to the A¹ substituent and the carbon attached to the A² substituent. In another embodiment, the compounds of formula Ie are at least 70% a single stereoisomer at both the carbon attached to the A¹ substituent and the carbon attached to the A² substituent. In another embodiment, the compounds of formula Ie are at least 80% a single stereoisomer at both the carbon attached to the A¹ substituent and the carbon attached to the A² substituent. In another embodiment, the compounds of formula Ie are at least 90% a single stereoisomer at both the carbon attached to the A¹ substituent and the carbon attached to the A² substituent. In another embodiment, the compounds of formula Ie are at least 95% a single stereoisomer at both the carbon attached to the A¹ substituent and the carbon attached to the A² substituent.

In one embodiment of the compounds of formula Ie, X is R^{1a}OC(O)—.

In another embodiment of the compounds of formula Ie, X is R^{1b}C(O)—.

In another embodiment of the compounds of formula Ie, X is R^{1b}S(O)₂—.

In another embodiment of the compounds of formula Ie, X is R^{1a}(R^{a})NC(O)—.

In another embodiment of the compounds of formula Ie, X is R^{1a}(R^{a})NS(O)₂—.

In another embodiment of the compounds of formula Ie, X is selected from the group consisting of R^{1a}OC(O)—, R^{1b}C(O)—, R^{1a}(R^{a})NC(O)— and R^{1b}S(O)₂—.

In one embodiment of the compounds of formula Ie, R^{a} is H.

In another embodiment of the compounds of formula Ie, R^{a} is (C₁-C₆)alkyl.

In one embodiment of the compounds of formula Ie, R^{1a} is (C₁-C₈)alkyl, (C₃-C₆)carbocyclyl or aryl(C₁-C₆)alkyl, wherein any (C₁-C₈)alkyl of R^{1a} is optionally substituted with one or more Z³ groups and wherein any (C₃-C₆)carbocyclyl or aryl(C₁-C₆)alkyl of R^{1a} is optionally substituted with one or more Z⁴ groups.

In another embodiment of the compounds of formula Ie, R^{1a} is propyl, butyl, pentyl, 2-methylpentyl, 2,5-dimethylhexyl, 2,2-dimethylpentyl, phenylpropyl, pentylcyclohexyl, (prop-2-yl)cyclohexyl, methylcyclohexyl or (4-chlorophenyl)cyclohexyl.

In another embodiment of the compounds of formula Ie, R^{1a} is propyl, butyl, pentyl, 2-methylpentyl, 2,5-dimethylhexyl, 2,2-dimethylpentyl, phenylpropyl, 4-pentylcyclohexyl, 4-(prop-2-yl)cyclohexyl, 4-methylcyclohexyl or 4-(4-chlorophenyl)cyclohexyl.

In another embodiment of the compounds of formula Ie, R^{1a} is prop-2-yl, but-2-yl, pent-3-yl, 4-methylpent-2-yl, 2,5-dimethylhex-3-yl, 4,4-dimethylpent-2-yl, 1-phenylprop-1-yl, 4-pentylcyclohexyl, 4-(prop-2-yl)cyclohexyl, 4-methylcyclohexyl or 4-(4-chlorophenyl)cyclohexyl.

In one embodiment of the compounds of formula Ie, R^{1b} is (C₁-C₈)alkyl, (C₃-C₆)carbocyclyl or aryl(C₁-C₆)alkyl, wherein any (C₁-C₈)alkyl of R^{1b} is optionally substituted with one or more Z³ groups and wherein any (C₃-C₆)carbocyclyl or aryl(C₁-C₆)alkyl of R^{1b} is optionally substituted with one or more Z⁴ groups.

In another embodiment of the compounds of formula Ie, R^{1b} is propyl, butyl, pentyl, 2-methylpentyl, 2,5-dimethylhexyl, 2,2-dimethylpentyl, phenylpropyl, pentylcyclohexyl, (prop-2-yl)cyclohexyl, methylcyclohexyl or (4-chlorophenyl)cyclohexyl.

In another embodiment of the compounds of formula Ie, R^{1b} is propyl, butyl, pentyl, 2-methylpentyl, 2,5-dimethylhexyl, 2,2-dimethylpentyl, phenylpropyl, 4-pentylcyclohexyl, 4-(prop-2-yl)cyclohexyl, 4-methylcyclohexyl or 4-(4-chlorophenyl)cyclohexyl.

In another embodiment of the compounds of formula Ie, R^{1b} is prop-2-yl, but-2-yl, pent-3-yl, 4-methylpent-2-yl, 2,5-dimethylhex-3-yl, 4,4-dimethylpent-2-yl, 1-phenylprop-1-yl, 4-pentylcyclohexyl, 4-(prop-2-yl)cyclohexyl, 4-methylcyclohexyl or 4-(4-chlorophenyl)cyclohexyl.

In one embodiment of the compounds of formula Ie, each Z⁴ group is independently selected from (C₁-C₆)alkyl and aryl, wherein any aryl of Z⁴ is optionally substituted with one or more halogen.

In another embodiment of the compounds of formula Ie, each Z⁴ group is independently selected from methyl, pentyl, isopropyl and 4-chlorophenyl.

In one embodiment of the compounds of formula Ie, A¹ is aryl(C₁-C₆)alkyl, wherein any aryl(C₁-C₆)alkyl of A¹ is optionally substituted with one or more Z² groups.

In another embodiment of the compounds of formula Ie, A¹ is aryl-CH₂—, wherein any aryl-CH₂— of A¹ is optionally substituted with one or more Z² groups.

In another embodiment of the compounds of formula Ie, A¹ is phenyl(C₁-C₆)alkyl, wherein any phenyl(C₁-C₆)alkyl of A¹ is optionally substituted with one or more Z² groups.

In another embodiment of the compounds of formula Ie, A¹ is phenyl-CH₂—, wherein any phenyl-CH₂— of A¹ is optionally substituted with one or more Z² groups.

In another embodiment of the compounds of formula Ie, A¹ is aryl-CH₂—.

In another embodiment of the compounds of formula Ie, A¹ is phenyl-CH₂—.

In one embodiment of the compounds of formula Ie, A² is aryl(C₁-C₆)alkyl, wherein any aryl(C₁-C₆)alkyl of A² is optionally substituted with one or more Z² groups.

In another embodiment of the compounds of formula Ie, A² is aryl-CH₂—, wherein any aryl-CH₂— of A² is optionally substituted with one or more Z² groups.

In another embodiment of the compounds of formula Ie, A² is phenyl(C₁-C₆)alkyl, wherein any phenyl(C₁-C₆)alkyl of A² is optionally substituted with one or more Z² groups.

In another embodiment of the compounds of formula Ie, A² is phenyl-CH₂—, wherein any phenyl-CH₂— of A² is optionally substituted with one or more Z² groups.

In another embodiment of the compounds of formula Ie, A² is aryl-CH₂—.

In another embodiment of the compounds of formula Ie, A² is phenyl-CH₂—.

In one embodiment of the compounds of formula Ie, Y is —C(O)O—.

In another embodiment of the compounds of formula Ie, Y is —C(O)N(R^{b})—.

In one embodiment of the compounds of formula Ie, R^{b} is H or (C₁-C₆)alkyl.

In another embodiment of the compounds of formula Ie, R^{b} is H.

In another embodiment of the compounds of formula Ie, R^{b} is (C₁-C₆)alkyl.

In one embodiment of the compounds of formula Ie, R⁴ is heteroaryl(C₁-C₆)alkyl,

wherein the heteroaryl(C₁-C₆)alkyl of R⁴ is optionally substituted with one or more Z⁵.

In another embodiment of the compounds of formula Ie, R⁴ is thiazolyl(C₁-C₆)alkyl, wherein thiazolyl is optionally substituted with one or more Z⁵ groups.

In another embodiment of the compounds of formula Ie, R⁴ is heteroaryl(C₁-C₆)alkyl.

In another embodiment of the compounds of formula Ie, R⁴ is heteroarylmethyl.

In another embodiment of the compounds of formula Ie, R⁴ is thiazolylmethyl.

In another embodiment of the compounds of formula Ie, R⁴ is thiazol-5-ylmethyl.

In one embodiment of the compounds of formula Ie, R² is H or (C₁-C₆)alkyl.

In another embodiment of the compounds of formula Ie, R² is H.

In another embodiment of the compounds of formula Ie, R² is (C₁-C₆)alkyl.

In one embodiment of the compounds of formula Ie, R³ is H or (C₁-C₆)alkyl.

In another embodiment of the compounds of formula Ie, R³ is H.

In another embodiment of the compounds of formula Ie, R³ is (C₁-C₆)alkyl.

In one embodiment, the compound of the invention has an inhibition activity against P450 at a level equal to or better than the inhibition activity of a compound as represented by an IC₅₀ of less than about 2000 nM, less than about 1500 nM, less than about 1000 nM, less than about 900 nM, less than about 800 nM, less than about 700 nM, less than about 650 nM, less than about 600 nM, less than about 550 nM, less than about 500 nM, less than about 400 nM, less than about 350 nM, less than about 300 nM, less than about 250 nM, less than about 200 nM, less than about 100 nM, or less than about 50 nM.

In another embodiment, the compound of the invention has an inhibition activity against an isozyme of P450, e.g., 3A in a range represented by IC₅₀ from about 2000 nM to about 100 nM, from about 1000 nM to about 100 nM, from about 900 nM to about 200 nM, from about 800 nM to about 300 nM, from about 700 nM to about 200 nM, from about 600 nM to about 200 nM, from about 500 nM to about 200 nM, from about 700 nM to about 300 nM, from about 600 nM to about 300 nM, from about 700 nM to about 400 nM, from about 600 nM to about 400 nM, from about 400 nM to about 100 nM, from about 300 nM to about 100 nM, or from about 600 nM to about 150 nM.

In another embodiment, the compound of the invention has an inhibition activity against P450 at a level equal to or better than the inhibition activity of a compound as represented by an IC₅₀ of less than about 2000 nM, less than about 1500 nM, less than about 1000 nM, less than about 900 nM, less than about 800 nM, less than about 700 nM, less than about 650 nM, less than about 600 nM, less than about 550 nM, less than about 500 nM, less than about 400 nM, less than about 350 nM, less than about 300 nM, less than about 250 nM, less than about 200 nM, less than about 100 nM, or less than about 50 nM, provided that such compound also does not substantially exhibit biological activities other than its inhibition activity against P450. For example, the compound of the invention can have a reduced or not significant activity of protease inhibition, including without any limitation a level of protease inhibition as represented by HIV EC₅₀ of greater than about 1000 nM, greater than about 900 nM, greater than about 800 nM, greater than about 700 nM, greater than about 600 nM, greater than about 500 nM, greater than about 400 nM, greater than about 300 nM, greater than about 200 nM, greater than about 100 nM, greater than about 50 nM, greater than about 40 nM, greater than about 30 nM, greater than about 20 nM, greater than about 10 nM, greater than about 5 nM, or greater than about 1 nM.

In another embodiment, the compound of the invention has an inhibition activity specifically against one or more isozymes of P450 including without limitation 1A2, 2B6, 2C8, 2C19, 2C9, 2D6, 2E1, and 3A4, 5, 7, etc.

In another embodiment, the compound of the invention has an inhibition activity specifically against an isozyme of P450 that is involved in metabolizing anti-viral drugs, e.g., indinavir, nelfinavir, ritonavir, saquinavir etc.

In another embodiment, the compound of the invention has an inhibition activity specifically against one or more isozymes of P450, but not the other(s). For example, the compound of the present invention can have an inhibition activity specifically against P450 3A, but a reduced, insubstantial, or minimum inhibition activity against another isozyme of P450, e.g., P450 2C9.

### Pharmaceutical Formulations

The compounds of this invention can be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986), herein incorporated by reference in its entirety. Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations of the invention, both for veterinary and for human use, comprise at least one active ingredient, e.g. a compound of the present invention, together with one or more acceptable carriers and optionally other therapeutic ingredients. The carrier(s) must be “acceptable” in the sense of being compatible with the other ingredients of the formulation and physiologically innocuous to the recipient thereof. The formulations include those suitable for the foregoing administration routes.

The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.), herein incorporated by reference in its entirety. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise one or more compounds of the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth herein, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 μm (including particle sizes in a range between 0.1 and 500 μm in increments such as 0.5 μm, 1 μM, 30 μm, 35 μm, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of infections as described herein.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients provided by the present invention the formulations of this invention may include other agents conventional in the art having regard to the type of formulation in question, for example those suitable for oral administration may include flavoring agents.

The invention further provides veterinary compositions comprising at least one active ingredient, e.g., a compound of the present invention together with a veterinary carrier.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

In one embodiment, the invention provides pharmaceutical compositions comprising a compound of the invention, or a pharmaceutically acceptable salt thereof, and a pharmaceutically acceptable carrier or excipient.

In another embodiment, the invention provides pharmaceutical compositions comprising a compound of the invention, or a pharmaceutically acceptable salt thereof, in combination with at least one additional therapeutic agent, and a pharmaceutically acceptable carrier or excipient.

### Combination Pharmaceutical Agent

Compounds of the invention can be combined with one or more additional therapeutic agents in a single composition to form a combination pharmaceutical agent.

According to the invention, the therapeutic agent used in combination with the compound of the invention can be any therapeutic agent having a therapeutic effect when used in combination with the compound of the invention. For example, the therapeutic agent used in combination with the compound of the invention can be any therapeutic agent that is accessible to oxidative metabolism by cytochrome P450 enzymes, especially cytochrome P450 monooxygenase, e.g., 1A2, 2B6, 2C8, 2C19, 2C9, 2D6, 2E1, 3A4,5,7, etc.

In one embodiment of the invention, the therapeutic agent used in combination with the compound of the invention can be any anti-viral agent, e.g., anti-HIV, anti-HCV, etc., anti-bacterial agent, anti-fungal agent, immuno-modulator, e.g., immunosuppressant, anti-neoplastic agent, chemotherapeutic agent, agents useful for treating cardiovascular conditions, neurological conditions, etc.

In another embodiment of the invention, the therapeutic agent used in combination with the compound of the invention can be any proton pump inhibitor, anti-epileptics, NSAID, oral hypoglycemic agent, angiotensin II, sulfonylureas, beta blocker, antidepressant, antipsychotics, or anesthetics, or a combination thereof.

In another embodiment, the invention provides a combination pharmaceutical agent comprising:

a) a first pharmaceutical composition comprising a compound of the invention, or a pharmaceutically acceptable salt thereof; and

b) a second pharmaceutical composition comprising at least one additional therapeutic agent selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, interferons, ribavirin analogs, NS3 protease inhibitors, alpha-glucosidase 1 inhibitors, hepatoprotectants, non-nucleoside inhibitors of HCV, and other drugs for treating HCV, and combinations thereof.

### Routes of Administration

One or more compounds of the invention are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally.

### Doses

The effective dose of an active ingredient depends at least on the nature of the condition being treated, toxicity, whether the compound is being used prophylactically (lower doses) or against an active disease or condition, the method of delivery, and the pharmaceutical formulation, and will be determined by the clinician using conventional dose escalation studies. The effective dose can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1000 mg, or between 5 mg and 500 mg, and may take the form of single or multiple doses.

### Co-Administration

Co-administration includes administration of any compound of the invention with one or more other active therapeutic agents in a single unitary dosage form (i.e. administration of a combination pharmaceutical agent).

Co-administration also includes administration of any compound of the invention as a unitary dosage form along with one or more other active therapeutic agents each in a unitary dosage form for simultaneous or sequential administration to a patient (i.e. combination therapy). Co-administration also includes administration of any compound of the invention with one or more active therapeutic agents as a unitary dosage form along with one or more active therapeutic agents each in a unitary dosage or optionally combined together to form a unitary dosage (or a combination thereof) for simultaneous or sequential administration to a patient (i.e. combination therapy). The unitary dosage forms (i.e. combination therapy) may be administered as a simultaneous or sequential regimen. When administered sequentially, the combination may be administered in two or more administrations.

Co-administration includes administration of unit dosages (as described above) of the compounds of the invention before or after administration of unit dosages of one or more other active therapeutic agents, for example, administration of the compounds of the invention within seconds, minutes, or hours of the administration of one or more other active therapeutic agents. For example, a unit dose of a compound of the invention can be administered first, followed within seconds or minutes by administration of a unit dose of one or more other active therapeutic agents. Alternatively, a unit dose of one or more other therapeutic agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound of the invention.

Co-administration of a compound of the invention with one or more other active therapeutic agents also refers to simultaneous or sequential administration of a compound of the invention and one or more other active therapeutic agents, such that therapeutically effective amounts of the compound of the invention and one or more other active therapeutic agents are both present in the body of the patient.

In one embodiment, the compounds of the invention can be used alone, e.g., for inhibiting cytochrome P450 monooxygenase. In another embodiment, the compounds of the present invention are used in combination with other active therapeutic ingredients or agents. Preferably, the other active therapeutic ingredients or agents are metabolized or accessible to the oxidative metabolism by cytochrome P450 enzymes, e.g., monooxygenase enzymes such as 1A2, 2B6, 2C8, 2C19, 2C9, 2D6, 2E1, 3A4,5,7, etc.

### Combinations

Combinations (for use in combination therapy) of the compounds of the present invention are typically selected based on the condition to be treated, cross-reactivities of ingredients and pharmaco-properties of the combination. For example, when treating an infection (e.g., HIV or HCV), the compositions of the invention are combined with anti-infective agents (such as those agents selected form the classes of compounds described herein).

In one embodiment, non-limiting examples of suitable combinations include combinations of one or more compounds of the present invention with one or more anti-viral agents, e.g., anti-HIV, anti-HCV, etc., anti-bacterial agents, anti-fungal agents, immuno-modulators, e.g., immunosuppressant, anti-neoplastic agents, chemotherapeutic agents, agents useful for treating cardiovascular conditions, neurological conditions, etc.

In another embodiment, non-limiting examples of suitable combinations include combinations of one or more compounds of the present invention with one or more proton pump inhibitors, anti-epileptics, NSAIDs, oral hypoglycemic agents, angiotensin II, sulfonylureas, beta blockers, antidepressants, antipsychotics, or anesthetics, or a combination thereof.

In another embodiment, non-limiting examples of suitable combinations include combinations of one or more compounds of the present invention with one or more HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, and other drugs for treating HIV, interferons, ribavirin analogs, HCV NS3 protease inhibitors, alpha-glucosidase 1 inhibitors, hepatoprotectants, nucleoside or nucleotide inhibitors of HCV, non-nucleoside inhibitors of HCV, and other drugs for treating HCV.

In another embodiment, the invention provides pharmaceutical compositions comprising a compound of the invention, or a pharmaceutically acceptable salt, thereof, in combination with at least one additional therapeutic agent selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, non-nucleoside inhibitors of HCV, CCR5 inhibitors, and combinations thereof, and a pharmaceutically acceptable carrier or excipient.

In another embodiment, the invention provides pharmaceutical compositions comprising a compound of the invention, or a pharmaceutically acceptable salt, thereof, in combination with at least one additional therapeutic agent selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, interferons, ribavirin analogs, NS3 protease inhibitors, alpha-glucosidase 1 inhibitors, hepatoprotectants, non-nucleoside inhibitors of HCV, and other drugs for treating HCV, and combinations thereof.

It is also contemplated that the compounds of invention can be used with any other active therapeutic agent or ingredient which is appreciably metabolized by cytochrome P450 monooxygenase enzymes, e.g. cytochrome P450 monooxygenase 3A, thereby reducing the amount or rate at which the other active therapeutic agent or ingredient is metabolized, whereby the pharmacokinetics of the other active therapeutic agent or ingredient is improved. The pharmacokinetics of a drug will determine the concentration of the drug at its intended site of therapeutic activity in an organism. Typical, but non-limiting, pharmacokinetic parameters measured are the half-life (t_{1/2}), maximum concentration (Cₘₐₓ), mean residence time (MRT), rate of clearance (CL) and volume of distribution (VD). Non-limiting examples of improved pharmacokinetic parameters would be increased t_{1/2}, increased MRT, increased Cₘₐₓ and decreased CL. In mammals, these parameters are usually determined by measuring the concentration of the drug in the blood over a period of time using conventional analytical techniques. Pharmacokinetic improvements usually include elevating the blood plasma levels of the other therapeutic agent or ingredient at a given time point or maintaining a therapeutically effective blood plasma level of the other therapeutic active agent or ingredient for a longer time period—compared to blood plasma levels of the other therapeutic agent or ingredient administered without the compound of the present invention. Although the blood may not be the optimal site of therapeutic activity for the drug, the concentration at the site of therapeutic activity is usually proportional to the concentration in the blood at a particular time point for a given dose of drug.

In another embodiment, the invention provides a method for improving the pharmacokinetics of a drug which is metabolized by cytochrome P450 monooxygenase, comprising administering to a patient treated with said drug, a therapeutically effective amount of a compound of the present invention, or a pharmaceutically acceptable salt thereof. In another aspect of this embodiment, the t_{1/2} is increased. In another aspect of this embodiment, the Cₘₐₓ is increased. In another aspect of this embodiment, the MRT is increased. In another aspect of this embodiment, the CL is decreased. In another aspect of this embodiment, co-administration of a therapeutically effective amount of a compound of formula I improves at least one of the pharmacokinetic parameters of the drug by at least about 10% to about 500%. In another aspect of this embodiment, co-administration of a therapeutically effective amount of a compound of formula I improves at least one of the pharmacokinetic parameters of the drug by at least about 10%. In another aspect of this embodiment, co-administration of a therapeutically effective amount of a compound of formula I improves at least one of the pharmacokinetic parameters of the drug by at least about 25%. In another aspect of this embodiment, co-administration of a therapeutically effective amount of a compound of formula I improves at least one of the pharmacokinetic parameters of the drug by at least about 50%. In another aspect of this embodiment, co-administration of a therapeutically effective amount of a compound of formula I improves at least one of the pharmacokinetic parameters of the drug by at least about 100%. In another aspect of this embodiment, co-administration of a therapeutically effective amount of a compound of formula I improves at least one of the pharmacokinetic parameters of the drug by at least about 200%. In another aspect of this embodiment, co-administration of a therapeutically effective amount of a compound of formula I improves at least one of the pharmacokinetic parameters of the drug by at least about 500%.

In another embodiment, the invention provides a method for improving the pharmacokinetics of a drug which is metabolized by cytochrome P450 monooxygenase, comprising administering to a patient treated with said drug, a therapeutically effective amount of a combination comprising said drug and a compound of the present invention, or a pharmaceutically acceptable salt thereof. In another aspect of this embodiment, the t_{1/2} is increased. In another aspect of this embodiment, the Cₘₐₓ is increased. In another aspect of this embodiment, the MRT is increased. In another aspect of this embodiment, the CL is decreased. In another aspect of this embodiment, the therapeutically effective amount of the combination improves at least one of the pharmacokinetic parameters of the drug by at least about 10% to about 500%. In another aspect of this embodiment, the therapeutically effective amount of the combination improves at least one of the pharmacokinetic parameters of the drug by at least about 10%. In another aspect of this embodiment, the therapeutically effective amount of the combination improves at least one of the pharmacokinetic parameters of the drug by at least about 25%. In another aspect of this embodiment, the therapeutically effective amount of the combination improves at least one of the pharmacokinetic parameters of the drug by at least about 50%. In another aspect of this embodiment, the therapeutically effective amount of the combination improves at least one of the pharmacokinetic parameters of the drug by at least about 100%. In another aspect of this embodiment, the therapeutically effective amount of the combination improves at least one of the pharmacokinetic parameters of the drug by at least about 200%. In another aspect of this embodiment, the therapeutically effective amount of the combination improves at least one of the pharmacokinetic parameters of the drug by at least about 500%.

In another embodiment, the invention provides a method for improving the pharmacokinetics of a drug which is metabolized by cytochrome P450 monooxygenase 3A, comprising administering to a patient treated with said drug, a therapeutically effective amount of a compound of the present invention, or a pharmaceutically acceptable salt thereof. In another aspect of this embodiment, the t_{1/2} is increased. In another aspect of this embodiment, the Cₘₐₓ is increased. In another aspect of this embodiment, the MRT is increased. In another aspect of this embodiment, the CL is decreased. In another aspect of this embodiment, co-administration of a therapeutically effective amount of a compound of formula I improves at least one of the pharmacokinetic parameters of the drug by at least about 10% to about 500%. In another aspect of this embodiment, co-administration of a therapeutically effective amount of a compound of formula I improves at least one of the pharmacokinetic parameters of the drug by at least about 10%. In another aspect of this embodiment, co-administration of a therapeutically effective amount of a compound of formula I improves at least one of the pharmacokinetic parameters of the drug by at least about 25%. In another aspect of this embodiment, co-administration of a therapeutically effective amount of a compound of formula I improves at least one of the pharmacokinetic parameters of the drug by at least about 50%. In another aspect of this embodiment, co-administration of a therapeutically effective amount of a compound of formula I improves at least one of the pharmacokinetic parameters of the drug by at least about 100%. In another aspect of this embodiment, co-administration of a therapeutically effective amount of a compound of formula I improves at least one of the pharmacokinetic parameters of the drug by at least about 200%. In another aspect of this embodiment, co-administration of a therapeutically effective amount of a compound of formula I improves at least one of the pharmacokinetic parameters of the drug by at least about 500%.

In another embodiment, the invention provides a method for increasing blood plasma levels of a drug which is metabolized by cytochrome P450 monooxygenase, comprising administering to a patient treated with said drug, a therapeutically effective amount of a compound of the invention, or a pharmaceutically acceptable salt thereof. In another aspect of this embodiment, co-administration of a therapeutically effective amount of a compound of formula I increases at least one of the blood plasma levels of the drug by at least about 10% to about 500%. In another aspect of this embodiment, co-administration of a therapeutically effective amount of a compound of formula I increases at least one of the blood plasma levels of the drug by at least about 10%. In another aspect of this embodiment, co-administration of a therapeutically effective amount of a compound of formula I increases at least one of the blood plasma levels of the drug by at least about 25%. In another aspect of this embodiment, co-administration of a therapeutically effective amount of a compound of formula I increases at least one of the blood plasma levels of the drug by at least about 50%. In another aspect of this embodiment, co-administration of a therapeutically effective amount of a compound of formula I increases at least one of the blood plasma levels of the drug by at least about 100%. In another aspect of this embodiment, co-administration of a therapeutically effective amount of a compound of formula I increases at least one of the blood plasma levels of the drug by at least about 200%. In another aspect of this embodiment, co-administration of a therapeutically effective amount of a compound of formula I increases at least one of the blood plasma levels of the drug by at least about 500%.

In another embodiment, the invention provides a method for increasing blood plasma levels of a drug which is metabolized by cytochrome P450 monooxygenase, comprising administering to a patient treated with said drug, a therapeutically effective amount of a combination comprising said drug and a compound of the present invention, or a pharmaceutically acceptable salt thereof. In another aspect of this embodiment, the therapeutically effective amount of the combination increases at least one of the blood plasma levels of the drug by at least about 10% to about 500%. In another aspect of this embodiment, the therapeutically effective amount of the combination increases at least one of the blood plasma levels of the drug by at least about 10%. In another aspect of this embodiment, the therapeutically effective amount of the combination increases at least one of the blood plasma levels of the drug by at least about 25%. In another aspect of this embodiment, the therapeutically effective amount of the combination increases at least one of the blood plasma levels of the drug by at least about 50%. In another aspect of this embodiment, the therapeutically effective amount of the combination increases at least one of the blood plasma levels of the drug by at least about 100%. In another aspect of this embodiment, the therapeutically effective amount of the combination increases at least one of the blood plasma levels of the drug by at least about 200%. In another aspect of this embodiment, the therapeutically effective amount of the combination increases at least one of the blood plasma levels of the drug by at least about 500%.

In another embodiment, the invention provides a method for increasing blood plasma levels of a drug which is metabolized by cytochrome P450 monooxygenase 3A, comprising administering to a patient treated with said drug, a therapeutically effective amount of a compound of the present invention, or a pharmaceutically acceptable salt thereof. In another aspect of this embodiment, co-administration of a therapeutically effective amount of a compound of formula I increases at least one of the blood plasma levels of the drug by at least about 10% to about 500%. In another aspect of this embodiment, co-administration of a therapeutically effective amount of a compound of formula I increases at least one of the blood plasma levels of the drug by at least about 10%. In another aspect of this embodiment, co-administration of a therapeutically effective amount of a compound of formula I increases at least one of the blood plasma levels of the drug by at least about 25%. In another aspect of this embodiment, co-administration of a therapeutically effective amount of a compound of formula I increases at least one of the blood plasma levels of the drug by at least about 50%. In another aspect of this embodiment, co-administration of a therapeutically effective amount of a compound of formula I increases at least one of the blood plasma levels of the drug by at least about 100%. In another aspect of this embodiment, co-administration of a therapeutically effective amount of a compound of formula I increases at least one of the blood plasma levels of the drug by at least about 200%. In another aspect of this embodiment, co-administration of a therapeutically effective amount of a compound of formula I increases at least one of the blood plasma levels of the drug by at least about 500%.

In another embodiment, the invention provides a method for increasing blood plasma levels of a drug which is metabolized by cytochrome P450 monooxygenase, comprising administering to a patient treated with said drug, a therapeutically effective amount of a compound of the invention, or a pharmaceutically acceptable salt thereof, and wherein the amount of the compound of the present invention administered is effective to inhibit cytochrome P450 monooxygenase. In another aspect of this embodiment, at least one of the blood plasma levels of the drug is increased by at least about 10% to about 500%. In another aspect of this embodiment, at least one of the blood plasma levels of the drug is increased by at least about 10%. In another aspect of this embodiment, at least one of the blood plasma levels of the drug is increased by at least about 25%. In another aspect of this embodiment, at least one of the blood plasma levels of the drug is increased by at least about 50%. In another aspect of this embodiment, at least one of the blood plasma levels of the drug is increased by at least about 100%. In another aspect of this embodiment, at least one of the blood plasma levels of the drug is increased by at least about 200%. In another aspect of this embodiment, at least one of the blood plasma levels of the drug is increased by at least about 500%.

In another embodiment, the invention provides a method for inhibiting cytochrome P450 monooxygenase in a patient comprising administering to a patient in need thereof an amount of a compound of the invention, or a pharmaceutically acceptable salt thereof, effective to inhibit cytochrome P450 monooxygenase.

In another embodiment, the invention provides a method for inhibiting cytochrome P450 monooxygenase 3A in a patient comprising administering to a patient in need thereof an amount of a compound of the invention, or a pharmaceutically acceptable salt thereof, effective to inhibit cytochrome P450 monooxygenase 3A.

In another embodiment, the invention provides a method for inhibiting cytochrome P450 monooxygenase comprising contacting cytochrome P450 monooxygenase with an amount of a compound of the invention, or a pharmaceutically acceptable salt thereof, effective to inhibit cytochrome P450 monooxygenase.

In another embodiment, the invention provides a method for inhibiting cytochrome P450 monooxygenase 3A comprising contacting cytochrome P450 monooxygenase 3A with an amount of a compound of the invention, or a pharmaceutically acceptable salt thereof, effective to inhibit cytochrome P450 monooxygenase 3A.

In another embodiment, the invention provides a method for treating an HIV infection comprising administering to a patient in need thereof a therapeutically effective amount of a compound of the present invention, or a pharmaceutically acceptable salt thereof, in combination with a therapeutically effective amount of one or more additional therapeutic agents selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, G6PD and NADH-oxidase inhibitors, CCR5 inhibitors and other drugs for treating HIV.

In another embodiment, the invention provides a method for treating an HCV infection comprising administering to a patient in need thereof a therapeutically effective amount of the present invention, or a pharmaceutically acceptable salt thereof, in combination with a therapeutically effective amount of one or more additional therapeutic agents selected from the group consisting of interferons, ribavirin analogs, NS3 protease inhibitors, alpha-glucosidase 1 inhibitors, hepatoprotectants, non-nucleoside inhibitors of HCV, and other drugs for treating HCV.

In another embodiment, the invention provides for the use of a compound of the invention, or a pharmaceutically acceptable salt thereof, for the preparation of a medicament for inhibiting cytochrome P450 monooxygenase in a patient.

In another embodiment, the invention provides for the use of a compound of the invention, or a pharmaceutically acceptable salt thereof, for the preparation of a medicament for inhibiting cytochrome P450 monooxygenase 3A in a patient.

### EXAMPLES

Exemplary methods for preparing the compounds of formula (I) are provided below. These methods are intended to illustrate the nature of such preparations and are not intended to limit the scope of applicable methods. While the examples specify certain reaction conditions, one skilled in the art will understand how to vary the specific reaction conditions to obtain the full scope of the invention.

In each of the exemplary schemes it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium, and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example; boiling point and molecular weight for distillation and sublimation, presence or absence of polar functional groups for chromatography, stability of materials in acidic and basic media in multiphase extractions; and the like. One skilled in the art will apply techniques most likely to achieve the desired separation.

It will be appreciated that synthetic intermediates may bear one or more protecting groups.

### EXAMPLES

### Example 1

### Preparation of Compound 2

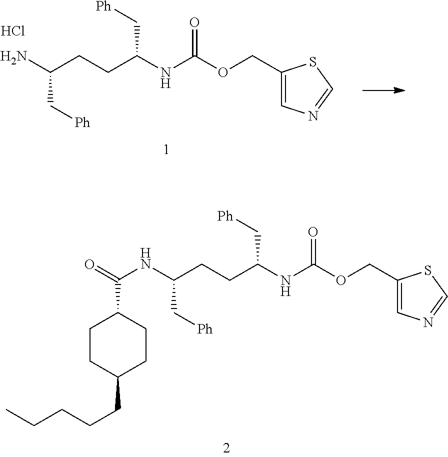 [SMILES: Cl ; N[C@H](CC[C@H](Cc1ccccc1)NC(=O)OCc1cncs1)Cc1ccccc1 ; CCCCC[C@H]1CC[C@H](C(=O)N[C@H](CC[C@H](Cc2ccccc2)NC(=O)OCc2cncs2)Cc2ccccc2)CC1]

To the stirred solution of compound 1 (compound 1 was prepared by the method of PCT/US2008/054788) (120 mg, 0.272 mmol) and 4-pentyl-cyclohexanecarboxylic acid (54 mg, 0.272 mmol) in DMF (2 mL) at room temperature was added HOBt (56 mg, 0.414 mmol), DIPEA (0.2 ml, 1.088 mmol) and EDC (0.10 ml, 0.454 mmol). The mixture was stirred for 16 hours. The solvents were removed, and the residue was diluted with EtOAc. The organic layer was washed with saturated NaHCO₃, water and brine, and dried over Na₇SO₄. Concentration and purification of the resulted crude material by column chromatography gave compound 2 (95 mg). ¹H-NMR (300 MHz, DMSO) δ 9.06 (s, 1H); 7.86 (s, 1H); 7.35 (d, J=8.4 Hz, 1H); 7.21-7.10 (m, 10H); 5.21 (s, 2H); 3.88-3.64 (m, 2H); 3.32-3.29 (m, 3H); 2.59-2.49 (m, 4H); 1.88-1.84 (m, 1H); 1.67 (s, 2H); 1.57-1.11 (m, 16H); 0.87-0.82 (m, 3H); m/z 590.3 (M+H)⁺.

### Example 2

### Preparation of Compound 3

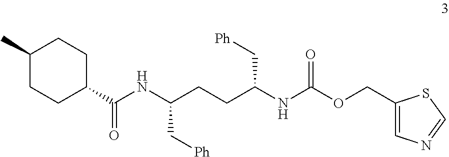 [SMILES: C[C@H]1CC[C@H](C(=O)N[C@H](CC[C@H](Cc2ccccc2)NC(=O)OCc2cncs2)Cc2ccccc2)CC1]

Compound 3 was prepared following the procedure used in Example 1 to prepare compound 2, except that 4-methyl-cyclohexanecarboxylic acid was used instead of 4-pentyl-cyclohexanecarboxylic acid. ¹H-NMR (300 MHz, DMSO) δ 9.07 (s, 1H); 7.87 (s, 1H); 7.36 (d, J=8.7 Hz, 1H); 7.21-7.10 (m, 10H); 5.17 (s, 2H); 3.89-3.65 (m, 2H); 2.61-2.60 (m, 4H); 1.90-1.86 (m, 1H); 1.61-1.17 (m, 11H); 0.84-0.82 (m, 5H); m/z 534.2 (M+H)⁺.

### Example 3

### Preparation of Compound 4

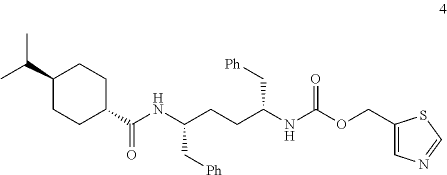 [SMILES: CC(C)[C@H]1CC[C@H](C(=O)N[C@H](CC[C@H](Cc2ccccc2)NC(=O)OCc2cncs2)Cc2ccccc2)CC1]

Compound 4 was prepared following the procedure used in Example 1 to prepare compound 2, except that 4-isopropyl-cyclohexanecarboxylic acid was used instead of 4-pentylcyclohexanecarboxylic acid. ¹H-NMR (300 MHz, DMSO) δ 9.06 (s, 1H); 7.86 (s, 1H); 7.34 (d, J=8.7 Hz, 1H); 7.21-7.10 (m, 10H); 5.16 (s, 2H); 3.88-3.65 (m, 2H); 2.61-2.59 (m, 4H); 1.88-1.84 (m, 1H); 1.61-1.14 (m, 10H); 0.92-0.88 (m, 2H); 0.83-0.80 (m, 6H) m/z 562.2 (M+H)⁺.

### Example 4

### Preparation of Compound 5

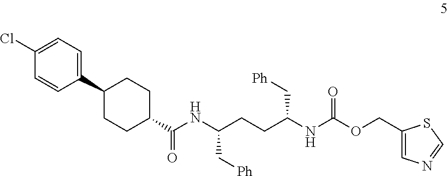 [SMILES: O=C(N[C@H](CC[C@H](Cc1ccccc1)NC(=O)[C@H]1CC[C@H](c2ccc(Cl)cc2)CC1)Cc1ccccc1)OCc1cncs1]

Compound 5 was prepared following the procedure used in Example 1 to prepare compound 2, except that 4-(4-chloro-phenyl)cyclohexanecarboxylic acid was used instead 4-pentylcyclohexanecarboxylic acid. ¹H-NMR (300 MHz, DMSO) δ 9.07 (s, 1H); 7.87 (s, 1H); 7.44 (d, J=8.7 Hz, 1H); 7.33-7.12 (m, 14H); 5.17 (s, 2H); 3.91-3.66 (m, 2H); 2.63-2.61 (m, 4H); 2.49 (s, 1H); 2.02 (s, 1H); 1.75-1.23 (m, 12H) m/z 630.3 (M+H)⁺.

### Example 5

### Preparation of Compound 6

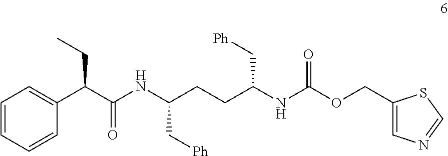 [SMILES: CC[C@@H](C(=O)N[C@H](CC[C@H](Cc1ccccc1)NC(=O)OCc1cncs1)Cc1ccccc1)c1ccccc1]

Compound 6 was prepared following the procedure used in Example 1 to prepare compound 2, except that 2-phenyl-butyric acid was used instead of 4-pentyl-cyclohexanecarboxylic acid. ¹H-NMR (300 MHz, DMSO) δ 9.07 (s, 1H); 7.87 (s, 1H); 7.75 (d, J=8.7 Hz, 1H); 7.23-7.04 (m, 15H); 5.16 (s, 2H); 3.92-3.65 (m, 2H); 3.25-3.21 (m, 1H); 2.60-2.50 (m, 4H); 1.90-1.50 (m, 2H); 1.42-1.37 (m, 4H); 0.78-0.75 (m, 3H) m/z 556.2 (M+H)⁺.

### Example 6

### Preparation of Compound 7

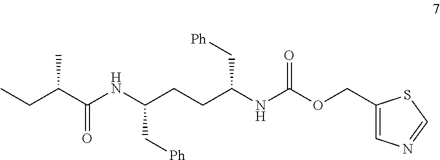 [SMILES: CC[C@H](C)C(=O)N[C@H](CC[C@H](Cc1ccccc1)NC(=O)OCc1cncs1)Cc1ccccc1]

Compound 7 was prepared following the procedure used in Example 1 to prepare compound 2, except that 2-methyl-butyric acid was used instead of 4-pentyl-cyclohexanecarboxylic acid. ¹H-NMR (300 MHz, DMSO) δ 9.06 (s, 1H); 7.88 (s, 1H); 7.43 (d, J=8.7 Hz, 1H); 7.21-7.10 (m, 10H); 5.16 (s, 2H); 3.93-3.67 (m, 2H); 2.62-2.53 (m, 4H); 2.01-1.99 (m, 1H); 1.40-1.38 (m, 4H); 1.21-1.15 (m, 2H); 0.79-0.67 (m, 6H) m/z 494.1 (M+H)⁺.

### Example 7

### Preparation of Compound 8

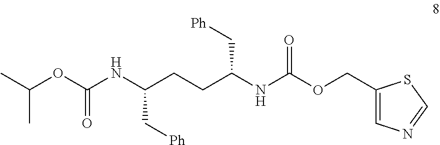 [SMILES: CC(C)OC(=O)N[C@H](CC[C@H](Cc1ccccc1)NC(=O)OCc1cncs1)Cc1ccccc1]

To the stirred solution of propan-2-ol (16 mg, 0.22 mmol) and KOH (cat.) in toluene (1 mL) was added CDI (40 mg, 0.24 mmol). The mixture was stirred at rt. for 16 hours. Removed the solvent and the residue was dissolved in 1 ml DMF. To this mixture was added a solution of compound 1 (100 mg, 0.22 mmol) in DMF (2 mL), and the mixture was heated at 110 C for additional 6 hours. The solvents were removed, and the residue was diluted with EtOAc. The organic layer was washed twice with water and once with brine, and dried over Na₂SO₄. Concentration and purification of the resulted crude material by column chromatography gave Compound 8 (55 mg). ¹H-NMR (300 MHz, DMSO) δ 9.06 (s, 1H), 7.86 (s, 1H), 7.22-7.10 (m, 10H); 6.77 (d, J=8.4 Hz, 1H); 5.16 (s, 2H); 4.64-4.60 (m, 1H); 3.62 (s, 2H); 2.63-2.58 (m, 4H); 1.37 (S, 4H); 1.11-1.03 (m, 6H); m/z 496.1 (M+H)⁺.

### Example 8

### Preparation of Compound 9

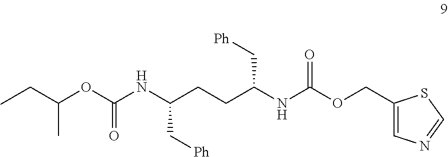 [SMILES: CCC(C)OC(=O)N[C@H](CC[C@H](Cc1ccccc1)NC(=O)OCc1cncs1)Cc1ccccc1]

Compound 9 was prepared following the procedure outlined in Example 7 used to prepare compound 8, except that butan-2-ol was used instead of propan-2-ol. ¹H-NMR (300 MHz, CD₃OD) δ 8.98 (s, 1H); 7.83 (s, 1H); 7.22-7.15 (m, 10H); 5.21 (s, 2H); 4.60-4.48 (m, 1H); 3.80 (s, 2H); 2.71-2.68 (m, 4H); 1.51-1.45 (m, 6H); 1.51-1.08 (m, 3H); 0.89-0.82 (m, 3H) m/z 510.0 (M+H)⁺.

### Example 9

### Preparation of Compound 10

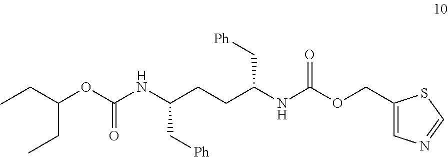 [SMILES: CCC(CC)OC(=O)N[C@H](CC[C@H](Cc1ccccc1)NC(=O)OCc1cncs1)Cc1ccccc1]

Compound 10 was prepared following the procedure outlined in Example 7 used to prepare compound 8, except that pentan-3-ol was used instead of propan-2-ol. ¹H-NMR (300 MHz, CD₃OD) δ 8.98 (s, 1H); 7.83 (s, 1H); 7.22-7.15 (m, 10H); 5.22 (s, 2H); 4.45-4.40 (m, 1H); 3.90 (s, 2H); 2.70-2.69 (m, 4H); 1.52-1.49 (m, 8H); 0.87-0.79 (m, 6H); m/z 524.0 (M+H)⁺.

### Example 10

### Preparation of Compound 11

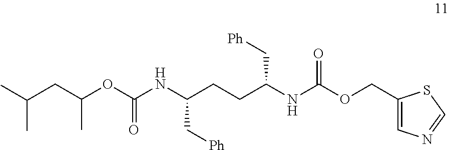 [SMILES: CC(C)CC(C)OC(=O)N[C@H](CC[C@H](Cc1ccccc1)NC(=O)OCc1cncs1)Cc1ccccc1]

Compound 11 was prepared following the procedure outlined in Example 7 used to prepare compound 8, except that 4-methyl-pentan-2-ol was used instead of propan-2-ol. ¹H-NMR (300 MHz, CD₃OD) δ 8.98 (s, 1H); 7.83 (s, 1H); 7.22-7.15 (m, 10 H); 5.22 (s, 2H); 4.80-4.70 (m, 1H); 3.80 (s, 2H); 2.72-2.64 (m, 4H); 1.52-1.41 (m, 6H); 1.28-1.05 (m, 4H); 0.91-0.81 (m, 6H) m/z 538.1 (M+H)⁺.

### Example 11

### Preparation of Compound 12

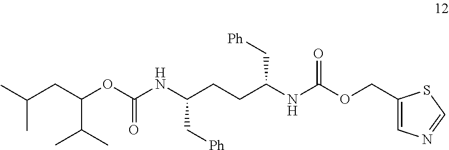 [SMILES: CC(C)CC(OC(=O)N[C@H](CC[C@H](Cc1ccccc1)NC(=O)OCc1cncs1)Cc1ccccc1)C(C)C]

Compound 12 was prepared following the procedure outlined in Example 7 used to prepare compound 8, except that 2,5-dimethyl-hexan-3-ol was used instead of propan-2-ol. ¹H-NMR (300 MHz, CD₃OD) δ 8.96 (s, 1H); 7.82 (s, 1H); 7.22-7.15 (m, 10 H); 5.21 (s, 2H); 4.57-4.53 (m, 1H); 3.81 (s, 2H); 2.75-2.61 (m, 4H); 1.64-1.10 (m, 8H); 0.91-0.78 (m, 12H) m/z 566.0 dg (M+H)⁺.

### Example 12

### Preparation of Compound 13

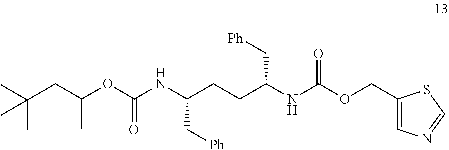 [SMILES: CC(CC(C)(C)C)OC(=O)N[C@H](CC[C@H](Cc1ccccc1)NC(=O)OCc1cncs1)Cc1ccccc1]

Compound 13 was prepared following the procedure outlined in Example 7 used to prepare compound 8, except that 4,4-dimethyl-pentan-2-ol was used instead of propan-2-ol. ¹H-NMR (300 MHz, CD₃OD) δ 8.97 (s, 1H); 7.83 (s, 1H); 7.25-7.14 (m, 10 H); 5.22 (s, 2H); 4.88-4.85 (m, 1H); 3.80 (s, 2H); 2.69-2.63 (m, 4H); 1.56-1.50 (m, 5H); 1.30-1.25 (m, 1H); 1.13-1.03 (m, 3H), 0.90-0.81 (m, 9H) m/z 552.1 (M+H)⁺.

### Example 13

### Preparation of Compound 14

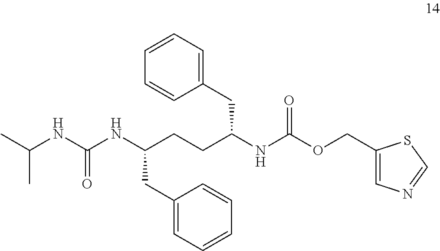 [SMILES: CC(C)NC(=O)N[C@H](CC[C@H](Cc1ccccc1)NC(=O)OCc1cncs1)Cc1ccccc1]

To the stirred solution of compound 1 (120 mg, 0.27 mmol) and diisopropylethylamine (187 μL, 1.08 mmol) in DMF (1 mL) was added CDI (48 mg, 0.3 mmol). The mixture was stirred for 16 hours. To this mixture was added a solution of isopropylamine (32 mg, 0.54 mmol) in DMF (1 mL), and the mixture was stirred for 5 additional hours. The solvents were removed, and the residue was diluted with EtOAc. The organic layer was washed twice with water and once with brine, and dried over Na₂SO₄. Concentration and purification by column chromatography gave Compound 14 (22 mg). ¹H-NMR (300 MHz, CD₃OD) δ 8.98 (s, 1H); 7.83 (s, 1H); 7.23-7.16 (m, 10H); 5.21 (s, 2H); 3.86-3.75 (m, 3H); 2.72-2.67 (m, 4H); 1.50-1.35 (m, 4H); 1.07-1.04 (m, 6H); m/z 495.1 (M+H)⁺.

### Example 14

### Preparation of Compound 15

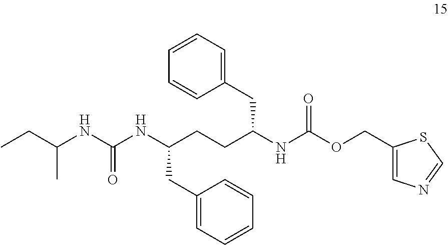 [SMILES: CCC(C)NC(=O)N[C@H](CC[C@H](Cc1ccccc1)NC(=O)OCc1cncs1)Cc1ccccc1]

Compound 15 was prepared following the procedure outlined in Example 13 used to prepare compound 14, except that sec-butylamine was used instead of isopropylamine. ¹H-NMR (300 MHz, CD₃OD) δ 8.98 (s, 1H); 7.83 (s, 1H); 7.21-7.15 (m, 10H); 5.21 (s, 2H); 3.84-3.73 (m, 2H); 3.54 (s, 1H); 2.72-2.67 (m, 4H); 1.52-1.39 (m, 6H); 1.04-0.85 (m, 6H); m/z 509.1 (M+H)⁺.

### Example 15

### Preparation of Compound 16

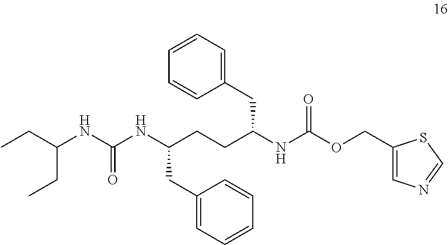 [SMILES: CCC(CC)NC(=O)N[C@H](CC[C@H](Cc1ccccc1)NC(=O)OCc1cncs1)Cc1ccccc1]

Compound 16 was prepared following the procedure outlined in Example 13 used to prepare compound 14, except that 1-ethyl-propylamine was used instead of isopropylamine. ¹H-NMR (300 MHz, CD₃OD) δ 8.98 (s, 1H); 7.83 (s, 1H); 7.21-7.15 (m, 10H); 5.21 (s, 2H); 3.84-3.73 (m, 2H); 3.54 (s, 1H); 2.72-2.67 (m, 4H); 1.52-1.39 (m, 8H); 0.85-0.82 (m, 6H); m/z 523.1 (M+H)⁺.

### Example 16

### Preparation of Compound 17

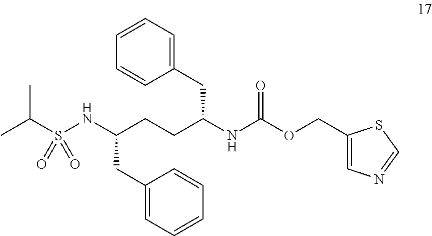 [SMILES: CC(C)S(=O)(=O)N[C@H](CC[C@H](Cc1ccccc1)NC(=O)OCc1cncs1)Cc1ccccc1]

To the mixture of compound 1 (120 mg, 0.272 mmol) in dichloromethane (2 mL) at 0° C. was added triethylamine (114 ul, 0.816 mmol) and propane-2-sulfonyl chloride (58 mg, 0.408 mmol). The mixture was stirred at room temperature for 16 hours. The solution was washed with water, brine, and dried over Na₂SO₄. Concentration and purification by column chromatography gave compound 17 (20 mg). ¹H-NMR (300 MHz, CD₃OD) δ 9.03 (s, 1H); 7.85 (s, 1H); 7.27-7.15 (m, 10H); 5.20 (s, 2H); 3.88-3.60 (m, 2H); 2.77-2.69 (m, 4H); 2.44-2.40 (m, 1H); 1.76-1.54 (m, 4H); 1.12-1.01 (m, 6H); m/z 516.1 (M+H)⁺.

### Example 17

### Preparation of Compound 18

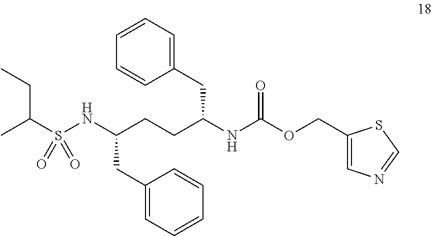 [SMILES: CCC(C)S(=O)(=O)N[C@H](CC[C@H](Cc1ccccc1)NC(=O)OCc1cncs1)Cc1ccccc1]

Compound 18 was prepared following the procedure outlined in Example 16 used to prepare compound 17 except that butane-2-sulfonyl chloride was used instead of propane-2-sulfonyl chloride. ¹H-NMR (300 MHz, CD₃OD) δ 8.96 (s, 1H); 7.81 (s, 1H); 7.28-7.19 (m, 10H); 5.20 (s, 2H); 3.88-3.64 (m, 2H); 2.82-2.61 (m, 4H); 2.31-2.23 (m, 1H); 1.75-1.67 (m, 2H); 1.60-1.51 (m, 4H); 1.13-0.98 (m, 6H); m/z 530.1 (M+H)⁺.

### IC₅₀ Determinations for Human Liver Cytochrome P450

### Materials and General Methods

Pooled (n≧15 donors) human hepatic microsomal fraction was obtained from BD-Gentest (Woburn, Mass.) who also supplied hydroxy-terfenadine, 4′-hydroxydiclofenac and NADPH regenerating system. Ritonavir was prepared from commercial Norvir® oral solution (Abbott Laboratories, Abbott Park, Ill.). Other reagents were from Sigma-Aldrich (St. Louis, Mo.) and included terfenadine, fexofenadine, BRL 15572, diclofenac and mefenamic acid.

Incubations were performed in duplicate in 50 mM potassium phosphate buffer, pH 7.4 with NADPH regenerating system used as described by the manufacturer. The final microsomal protein concentrations had previously been determined to be within the linear range for activity and resulted in less than 20% consumption of substrate over the course of the incubation. The final substrate concentrations used were equal to the apparent Km values for the activities determined under the same conditions. Inhibitors were dissolved in DMSO, and the final concentration of DMSO, from both substrate and inhibitor vehicles, was 1% (v/v). Incubations were performed at 37° C. with shaking and were initiated by addition of substrate. Aliquots were then removed at 0, 7 and 15 minutes. Samples were quenched by treatment with an acetonitrile, formic acid, water (94.8%/0.2%/5%, v/v/v) mixture containing internal standard. Precipitated protein was removed by centrifugation at 3000 rpm for 10 min and aliquots of the supernatant were then subjected to LC-MS analysis.

The LC-MS system consisted of a Waters Acquity UPLC, with a binary solvent manager and a refrigerated (8° C.) sample organizer and sample manager, interfaced to a Micromass Quattro Premier tandem mass spectrometer operating in electrospray ionization mode. The column was a Waters Acquity UPLC BEH C₁₈ 2.1×50 mm, 1.7 μm pore size. Mobile phases consisted of mixtures of acetonitrile, formic acid and water, the composition for mobile phase A being 1%/0.2%/98.8% (v/v/v) and that for mobile phase B being 94.8%/0.2%/5% (v/v/v). The injection volumes were 5 μL and the flow rate was 0.8 mL/min. Concentrations of metabolites were determined by reference to standard curves generated with authentic analytes under the same conditions as the incubations.

IC₅₀ values (the concentration of inhibitor reducing CYP3A activity by 50%) were calculated by non-linear regression using GraphPad Prism 4.0 software and a sigmoidal model.

### CYP3A Inhibition Assay

The potencies of the compounds as inhibitors of human hepatic cytochromes P450 of the CYP3A subfamily (particularly CYP3A4) were assessed using well-characterized selective activities: midazolam 1-hydroxylase (Kronbach, T., et al. Mol. Pharmacol. 36, 89-96 [1989]) and testosterone 6β-hydroxylase (Waxman, D. J., et al. Arch. Biochem. Biophys. 263, 424-436, [1988]). For midazolam hydroxylase determination the final concentrations of microsomal protein and terfenadine substrate were 0.25 mg/mL and 2.5 μM, respectively, and the LC-MS internal standard was 1α-hydroxytriazolam. For testosterone hydroxylase activity the final microsomal protein concentration was 0.5 mg/mL, the substrate concentration was 50 μM and the LC-MS internal standard was D7-labeled 6β-hydroxytestosterone. After incubation at 37° C. for 5 minutes, metabolic reactions were terminated by treatment with quench solution containing the appropriate internal standard and were subsequently centrifuged before aliquots of the supernatant were removed and diluted with 0.1% (v/v) formic acid for LC-MS analysis.

For LC-MS analysis the column was a Phenomenex Synergi Max RP 4 μm column (50×2.0 mm) and the injection volume was 5 μL. The MS/MS ion current metabolite/internal standard peak area ratios (PAR) were measured on an Applied Biosystems SciEx API4000 triple quadrupole mass spectrometer coupled to a Leap CTC PAL autosampler with a 20 μL loop and a Shimadzu LC pump with a 25 μL mixer. The initial mobile phase consisted of 94.9% water, 0.1% formic acid, and 5% acetonitrile (v/v/v) pumped at 0.5 mL/min. After 0.5 min the acetonitrile concentration was increased to 30% over 0.5 min. The acetonitrile concentration was then further increased to 100% over 0.5 min and held for 1.3 min, after which the column was re equilibrated at the initial conditions for 1.2 min. Rates of metabolite formation were determined from standard curves using PAR determined with authentic metabolite standards.

Experimental data based on representative Examples demonstrate that the compounds of formula I of the present invention have CYP3A4 inhibition activity in a range represented by an IC₅₀ from <100 nM to >25 Table I shows the activity of representative examples of the compounds of the invention for inhibition of CYP3A4.

### CYP2C9 Inhibition Assay

The potencies of the compounds as inhibitors of human hepatic CYP2C9 were assessed using tolbutamide-4-hydroxylase as a well-characterized selective activity (Miners, J. O., et al. Biochem. Pharmacol. 37, 1137-1144 [1988]). The final concentrations of microsomal protein and tolbutamide substrate were 1.0 mg/mL and 120 μM, respectively and reactions were initiated by the addition of NADPH to 1 mM. Sulfaphenazole, a positive control CYP2C9 inhibitor was tested in parallel. After incubation at 37° C. for 60 minutes, metabolic reactions were terminated by treatment with quench solution and were subsequently centrifuged before aliquots of the supernatant were removed and diluted with 0.1% (v/v) formic acid for LC-MS analysis.

For LC-MS analysis the column was a Phenomenex Synergi Max RP 4 μm column (50×2.0 mm) and the injection volume was 5 μL. The MS/MS ion current metabolite/internal standard peak area ratios (PAR) were measured on an Applied Biosystems SciEx API4000 triple quadrupole mass spectrometer coupled to a Leap CTC PAL autosampler with a 20 μL loop and a Shimadzu LC pump with a 25 μL mixer. The initial mobile phase consisted of 94.9% water, 0.1% formic acid, and 5% acetonitrile (v/v/v) pumped at 0.5 mL/min. After 0.5 min the acetonitrile concentration was increased to 30% over 0.5 min. The acetonitrile concentration was then further increased to 100% over 0.5 min and held for 1.3 min, after which the column was re equilibrated at the initial conditions for 1.2 min. Rates of metabolite formation were determined from standard curves using PAR determined with authentic 4-hydroxytolbutamide metabolite standard.

Experimental data based on representative Examples demonstrate that the compounds of formula I of the present invention have CYP2C9 inhibition activity in a range represented by an IC₅₀ from about 1 μM to >25 μM.

The following biological assays were used for characterizing representative compounds of the invention.

### HIV-1 Protease Enzyme Assay (Ki)

The assay is based on the fluorimetric detection of synthetic hexapeptide substrate cleavage by HIV-1 protease in a defined reaction buffer as initially described by M. V. Toth and G. R. Marshall, Int. J. Peptide Protein Res. 36, 544 (1990) (herein incorporated by reference in its entirety for all purposes).

The assay employed (2-aminobenzoyl)Thr-Ile-Nle-(p-nitro)Phe-Gln-Arg as the substrate and recombinant HIV-1 protease expressed in E. Coli as the enzyme. Both of the reagents were supplied by Bachem California, Inc. (Torrance, Calif.; Cat. no. H-2992). The buffer for this reaction was 100 mM ammonium acetate, pH 5.3, 1 M sodium chloride, 1 mM ethylendiaminetetraacetic acid, 1 mM dithiothreitol, and 10% dimethylsulfoxide.

To determine the inhibition constant Ki, a series of solutions were prepared containing identical amount of the enzyme (1 to 2.5 nM) and the inhibitor to be tested at different concentrations in the reaction buffer. The solutions were subsequently transferred into a white 96-well plate (190 μl each) and pre-incubated for 15 min at 37° C. The substrate was solubilized in 100% dimethylsulfoxide at a concentration of 800 μM and 10 μl of 800 μM substrate was added into each well to reach a final substrate concentration of 40 μM. The real-time reaction kinetics was measured at 37° C. using a Gemini 96-well plate fluorimeter (Molecular Devices, Sunnyvale, Calif.) at λ(Ex)=330 nm and λ(Em)=420 nm. Initial velocities of the reactions with different inhibitor concentrations were determined and the Ki value (in picomolar concentration units) was calculated by using EnzFitter program (Biosoft, Cambridge, U.K.) according to an algorithm for tight-binding competitive inhibition described by Ermolieff J., Lin X., and Tang J., Biochemistry 36, 12364 (1997).

### HIV-1 Protease Enzyme Assay (IC50)

As for the K assay, above, the IC₅₀ assay is based on the fluorimetric detection of synthetic hexapeptide substrate cleavage by HIV-1 protease in a defined reaction buffer as initially described by M. V. Toth and G. R. Marshall, Int. J. Peptide Protein Res. 36, 544 (1990).

The assay employed (2-aminobenzoyl)Thr-Ile-Nle-(p-nitro)Phe-Gln-Arg as the substrate and recombinant HIV-1 protease expressed in E. Coli as the enzyme. Both of the reagents were supplied by Bachem California, Inc. (Torrance, Calif.; Cat. nos. H-2992 and H-9040, respectively). The buffer for this reaction was 100 mM ammonium acetate, pH 5.5, 1 M sodium chloride, 1 mM ethylendiaminetetraacetic acid, and 1 mM dithiothreitol, and 10% dimethylsulfoxide.

To determine the IC50 value, 170 μL of reaction buffer was transferred into the wells of a white 96-well microtiter plate. A series of 3-fold dilutions in DMSO of the inhibitor to be tested was prepared, and 10 μL of the resulting dilutions was transferred into the wells of the microtiter plate. 10 μL of a 20-50 nM enzyme stock solution in reaction buffer was added to each well of the 96-well plate to provide a final enzyme concentration of 1-2.5 nM. The plates were then preincubated for 10 minutes at 37° C. The substrate was solubilzed in 100% dimethylsulfoxide at a concentration of 400 μM and 10 μl of the 400 μM substrate was added into each well to reach a final substrate concentration of 20 μM. The real-time reaction kinetics were measured using a Gemini 96-well plate fluorimeter (Molecular Devices, Sunnyvale, Calif.) at λ(Ex)=330 nm and λ(Em)=420 nm. Initial velocities of the reactions with different inhibitor concentrations were determined and the IC₅₀ value (in nanomolar concentration units) was calculated by using GraphPad Prism™ software to fit nonlinear regression curves.

### Anti-HIV-1 Cell Culture Assay (EC50)

The assay is based on quantification of the HIV-1-associated cytopathic effect by a colorimetric detection of the viability of virus-infected cells in the presence or absence of tested inhibitors. HIV-1-induced cell death was determined using a metabolic substrate 2,3-bis(2-methoxy-4-nitro-5-sulfophenyl)-2H-tetrazolium-5-carboxanilide (XTT) which is converted only by intact cells into a product with specific absorption characteristics as described by Weislow O S, Kiser R, Fine D L, Bader J, Shoemaker R H and Boyd M R, J. Natl. Cancer Inst. 81, 577 (1989) (herein incorporated by reference in its entirety for all purposes).

MT2 cells (NIH AIDS reagent program, Cat #237) maintained in RPMI-1640 medium supplemented with 5% fetal bovine serum and antibiotics were infected with the wild-type HIV-1 strain IIIB (Advanced Biotechnologies, Columbia, Md.) for 3 hours at 37° C. using the virus inoculum corresponding to a multiplicity of infection equal to 0.01. The infected cells in culture media were distributed into a 96-well plate (20,000 cells in 100 μl/well), and incubated in the presence of a set of solutions containing 5-fold serial dilutions of the tested inhibitor (100 μl/well) for 5 days at 37° C. Samples with untreated infected and untreated mock-infected control cells were also distributed to the 96-well plate and incubated under the same conditions.

To determine the antiviral activity of the tested inhibitors, a substrate XTT solution (6 mL per assay plate) at a concentration of 2 mg/mL in a phosphate-buffered saline pH 7.4 was heated in water-bath for 5 min at 55° C. before 50 μl of N-methylphenazonium methasulfate (5 μg/mL) was added per 6 mL of XTT solution. After removing 100 μl media from each well on the assay plate, 100 μl of the XTT substrate solution was added to each well. The cells and the XTT solution were incubated at 37° C. for 45 to 60 min in a CO₂ incubator. To inactivate the virus, 20 μl of 2% Triton X-100 was added to each well. Viability, as determined by the amount of XTT metabolites produced, was quantified spectrophotometrically by the absorbance at 450 nm (with subtraction of the background absorbance at 650 nm). Data from the assay was expressed as the percentage absorbance relative to untreated control and the fifty percent effective concentration (EC₅₀) was calculated as the concentration of compound that effected an increase in the percentage of XTT metabolite production in infected, compound treated cells to 50% of that produced by uninfected, compound-free cells.

### Anti-HIV-1 Cell Culture Assay (EC₅₀) in Presence of 40% Human Serum or Human Serum Proteins

This assay is almost identical to the Anti-HIV-1 Cell Culture Assay described above, except that the infection was made in the presence or absence of 40% human serum (Type AB Male Cambrex 14-498E) or human serum proteins (Human a-acid Glycoprotein, Sigma G-9885; Human Serum Albumin, Sigma A1653, 96-99%) at physiological concentration. The HIV-1-induced cell death was determined as described above, except that the infected cells distributed in the 96-well plate were incubated in 80% Human Serum (2× concentration) or in 2 mg/mL Human α-acid Glycoprotein+70 mg/mL HSA (2× concentration) rather than in culture media.

### Cytotoxicity Cell Culture Assay (CC₅₀)

The assay is based on the evaluation of cytotoxic effect of tested compounds using a metabolic substrate 2,3-bis(2-methoxy-4-nitro-5-sulfophenyl)-2H-tetrazolium-5-carboxanilide (XTT) as described by Weislow O S, Kiser R, Fine D L, Bader J, Shoemaker R H and Boyd M R, J. Natl. Cancer Inst. 81, 577 (1989). This assay is almost identical to the previous assay described (Anti-HIV-1 Cell Culture Assay), except that the cells were not infected. The compound induced cell death (or growth reduction) was determined as previously described.

MT-2 cells maintained in RPMI-1640 medium supplemented with 5% fetal bovine serum and antibiotics were distributed into a 96-well plate (20,000 cells in 100 Owen) and incubated in the presence or absence of 5-fold serial dilutions of the tested inhibitor (100 μl/well) for 5 days at 37° C. Controls included untreated infected cells and infected cells protected by 1 μM of P4405 (Podophyllotoxin, Sigma Cat # P4405).

To determine cytotoxicity, an XTT solution (6 mL per assay plate) at a concentration of 2 mg/mL in phosphate-buffered saline pH 7.4 was heated in the dark in a water-bath for 5 min at 55° C. before 50 μl of N-methylphenazonium methasulfate (5 μg/mL) was added per 6 mL of XTT solution. After removing 100 μL media from each well on the assay plate, 100 μL of the XTT substrate solution was added to each well. The cells and the XTT solution were incubated at 37° C. for 45 to 60 min in a CO₂ incubator. To inactivate the virus, 20 μl of 2% Triton X-100 was added to each well. Viability, as determined by the amount of XTT metabolites produced, is quantified spectrophotometrically by the absorbance at 450 nm (with subtraction of the background absorbance at 650 nm). Data from the assay is expressed as the percentage absorbance relative to untreated control, and the fifty percent cytotoxicity concentration (EC₅₀) was calculated as the concentration of compound that affected an increase in the percentage of cell growth in compound treated cells to 50% of the cell growth provided by uninfected, compound-free cells.

Experimental data based on representative Examples demonstrate that the compounds of formula I of the present invention have protease inhibition activity as represented by HIV EC₅₀ of greater than 25 μM

TABLE I
Activity of representative compounds of the invention against CYP3A4

| Compound | CYP3A4 IC₅₀ (μM) | CYP3A4 IC₅₀ (μM) |
|---|---|---|
| number | (midazolam) | (testosterone) |
| 2 | 2 | 10 |
| 3 | 0.12 | 0.27 |
| 4 | <0.1 | 3.4 |
| 5 | 1.8 | >25 |
| 6 | 0.51 | 1.4 |
| 7 | <0.1 | 0.36 |
| 8 | <0.1 | 0.38 |
| 9 | 0.13 | 0.43 |
| 10 | 0.20 | 0.50 |
| 11 | 0.25 | 0.71 |
| 12 | 0.38 | 0.40 |
| 13 | 0.40 | 0.54 |
| 14 | <0.1 | 0.27 |
| 15 | <0.1 | 0.25 |
| 16 | <0.1 | 0.41 |
| 17 | <0.1 | 0.32 |
| 18 | <0.1 | 0.34 |

What is claimed is:

1. A compound of formula I: 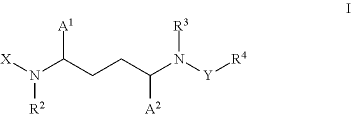
wherein:
A¹ is (C₁-C₆)alkyl, aryl(C₁-C₆)alkyl, heteroaryl(C₁-C₆)alkyl, (C₃-C₆)carbocyclyl(C₁-C₆)alkyl or heterocyclyl(C₁-C₆)alkyl, wherein any (C₁-C₆)alkyl of A¹ is optionally substituted with one or more Z¹ groups and wherein any aryl(C₁-C₆)alkyl, heteroaryl(C₁-C₆)alkyl, (C₃-C₆)carbocyclyl(C₁-C₆)alkyl or heterocyclyl(C₁-C₆)alkyl of A¹ is optionally substituted with one or more Z² groups;
A² is (C₁-C₆)alkyl, aryl(C₁-C₆)alkyl, heteroaryl(C₁-C₆)alkyl, (C₃-C₆)carbocyclyl(C₁-C₆)alkyl or heterocyclyl(C₁-C₆)alkyl, wherein any (C₁-C₆)alkyl of A² is optionally substituted with one or more Z¹ groups and wherein any aryl(C₁-C₆)alkyl, heteroaryl(C₁-C₆)alkyl, (C₃-C₆)carbocyclyl(C₁-C₆)alkyl or heterocyclyl(C₁-C₆)alkyl of A² is optionally substituted with one or more Z² groups;
X is selected from the group consisting of R^{1a}OC(O)—, R^{1b}C(O)—, R^{1a}(R^{a})NC(O)—, R^{1b}S(O)₂— and R^{1a}(R^{a})NS(O)₂—;
Y is —C(O)O— or —C(O)NR^{b}—;
R^{1a} is (C₁-C₈)alkyl, (C₃-C₆)carbocyclyl, aryl or aryl(C₁-C₆)alkyl, wherein any (C₁-C₈)alkyl of R^{1a} is optionally substituted with one or more Z³ groups and wherein any (C₃-C₆)carbocyclyl, aryl or aryl(C₁-C₆)alkyl of R^{1a} is optionally substituted with one or more Z⁴ groups;
R^{1b} is (C₁-C₈)alkyl, (C₃-C₆)carbocyclyl, aryl, aryl(C₁-C₆)alkyl, heteroaryl, heteroaryl(C₁-C₆)alkyl, heterocyclyl or heterocyclyl(C₁-C₆)alkyl, wherein any (C₁-C₈)alkyl of R^{1b} is optionally substituted with one or more Z³ groups and wherein any (C₃-C₆)carbocyclyl, aryl, aryl(C₁-C₆)alkyl, heteroaryl, heteroaryl(C₁-C₆)alkyl, heterocyclyl or heterocyclyl(C₁-C₆)alkyl of R^{1b} is optionally substituted with one or more Z⁴ groups;
R² is selected from the group consisting of H, (C₁-C₆)alkyl, (C₃-C₆)carbocyclyl, (C₃-C₆)carbocyclyl(C₁-C₆)alkyl- and aryl(C₁-C₆)alkyl;
R³ is selected from the group consisting of H, (C₁-C₆)alkyl, (C₃-C₆)carbocyclyl, C₃-C₆)carbocyclyl(C₁-C₆)alkyl- and aryl(C₁-C₆)alkyl;
R⁴ is aryl, aryl(C₁-C₆)alkyl, heteroaryl, heteroaryl(C₁-C₆)alkyl, heterocyclyl or heterocyclyl(C₁-C₆)alkyl, wherein any aryl, aryl(C₁-C₆)alkyl, heteroaryl, heteroaryl(C₁-C₆)alkyl, heterocyclyl or heterocyclyl(C₁-C₆)alkyl of R⁴ is optionally substituted with one or more Z⁵ groups;
each Z¹ is independently selected from OH, oxo, halogen, OCF₃, CN, —O(C₁-C₆)alkyl, S(C₁-C₆)alkyl, SO(C₁-C₆)alkyl, S(O)₂(C₁-C₆)alkyl, —NR^{c}R^{d}, —NRⁱC(O)R^{j} and —NRⁱS(O)₂R^{j};
each Z² is independently selected from OH, oxo, halogen, CF₃, OCF₃, NO₂, CN, (C₁-C₆)alkyl, —O(C₁-C₆)alkyl, S(C₁-C₆)alkyl, SO(C₁-C₆)alkyl, S(O)₂(C₁-C₆)alkyl, —NR^{c}R^{d}, —NRⁱC(O)R^{j} and —NRⁱS(O)₂R^{j};
each Z³ is independently selected from OH, halogen, CN, —O(C₁-C₆)alkyl, and —NR^{e}R^{f};
each Z⁴ is independently selected from OH, halogen, CF₃, OCF₃, NO₂, CN, (C₁-C₆)alkyl, —O(C₁-C₆)alkyl, aryl and —NR^{e}R^{f}, wherein any aryl of Z⁴ is optionally substituted with one or more (e.g. 1, 2, 3, 4 or 5) groups selected from OH, halogen, —CF₃, —OCF₃, NO₂, CN, (C₁-C₆)alkyl and —O(C₁-C₆)alkyl;
each Z⁵ is independently selected from OH, oxo, halogen, CF₃, OCF₃, NO₂, CN, (C₁-C₆)alkyl, —O(C₁-C₆)alkyl, and —NR^{g}R^{h};
R^{a} is selected from the group consisting of H, (C₁-C₆)alkyl and aryl(C₁-C₆)alkyl;
R^{b} is selected from the group consisting of H, (C₁-C₆)alkyl and aryl(C₁-C₆)alkyl;
R^{c} and R^{d} are each independently selected from H and (C₁-C₆)alkyl; or R^{c} and R^{d} together with the nitrogen to which they are attached form a piperidine, morpholine, piperazine, N-methylpiperazine or pyrrolidine;
R^{e} and R^{f} are each independently selected from H and (C₁-C₆)alkyl; or R^{e} and R^{f} together with the nitrogen to which they are attached form a piperidine, morpholine, piperazine, N-methylpiperazine or pyrrolidine; and
R^{g} and R^{h} are each independently selected from H and (C₁-C₆)alkyl; or R^{g} and R^{h} together with the nitrogen to which they are attached form a piperidine, morpholine, piperazine, N-methylpiperazine or pyrrolidine;
Rⁱ is H or (C₁-C₆)alkyl; and
R^{j} is (C₁-C₆)alkyl;
or a salt thereof.

2. The compound of claim 1 wherein X is R^{1a}(R^{a})NC(O)—.

3. The compound of claim 1 or claim 2 wherein R^{a} is H.

4. The compound of claim 1 wherein X is R^{1a}OC(O)—.

5. The compound of claim 1 wherein X is R^{1b}C(O)—.

6. The compound of claim 1 wherein X is R^{1b}S(O)₂—.

7. The compound of any one of claims 1-6 wherein each R^{1a} or R^{1b} is independently (C₁-C₈)alkyl, (C₃-C₆)carbocyclyl or aryl(C₁-C₆)alkyl, wherein any (C₁-C₈)alkyl of R^{1a} or R^{1b} is optionally substituted with one or more Z³ groups and wherein any (C₃-C₆)carbocyclyl or aryl(C₁-C₆)alkyl of R^{1a} or R^{1b} is optionally substituted with one or more Z⁴ groups.

8. The compound of any one of claims 1-7 wherein each Z⁴ group is independently selected from (C₁-C₆)alkyl and aryl, wherein any aryl of Z⁴ is optionally substituted with one or more halogen.

9. The compound of any one of claims 1-6 wherein each R^{1a} or R^{1b} is independently propyl, butyl, pentyl, 2-methylpentyl, 2,5-dimethylhexyl, 2,2-dimethylpentyl, or phenylpropyl, 4-pentylcyclohexyl, 4-(prop-2-yl)cyclohexyl, 4-methylcyclohexyl or 4-(4-chlorophenyl)cyclohexyl.

10. The compound of any one of claims 1-6 wherein each R^{1a} or R^{1b} is independently prop-2-yl, but-2-yl, pent-3-yl, 4-methylpent-2-yl, 2,5-dimethylhex-3-yl, 4,4-dimethylpent-2-yl, 1-phenylprop-1-yl, 4-pentylcyclohexyl, 4-(prop-2-yl)cyclohexyl, 4-methylcyclohexyl or 4-(4-chlorophenyl)cyclohexyl.

11. The compound of any one of claims 1-10 wherein A¹ is aryl(C₁-C₆)alkyl, wherein any aryl(C₁-C₆)alkyl of A¹ is optionally substituted with one or more Z² groups.

12. The compound of any one of claims 1-10 wherein A¹ is aryl-CH₂—, wherein any aryl-CH₂— of A¹ is optionally substituted with one or more Z² groups.

13. The compound of any one of claims 1-10 wherein A¹ is phenyl-CH₂—.

14. The compound of any one of claims 1-13 wherein A² is aryl(C₁-C₆)alkyl, wherein any aryl(C₁-C₆)alkyl of A² is optionally substituted with one or more Z² groups.

15. The compound of any one of claims 1-13 wherein A² is aryl-CH₂—, wherein any aryl-CH₂— of A² is optionally substituted with one or more Z² groups.

16. The compound of any one of claims 1-13 wherein A² is phenyl-CH₂—.

17. The compound of any one of claims 1-16 wherein Y is —C(O)O—.

18. The compound of any one of claims 1-17 wherein R⁴ is heteroaryl(C₁-C₆)alkyl, wherein any heteroaryl(C₁-C₆)alkyl of R⁴ is optionally substituted with one or more Z⁵ groups.

19. The compound of any one of claims 1-17 wherein R⁴ is thiazolyl(C₁-C₆)alkyl, wherein thiazolyl(C₁-C₆)alkyl is optionally substituted with one or more Z⁵ groups.

20. The compound of any one of claims 1-17 wherein R⁴ is thiazol-5-ylmethyl.

21. The compound of any one of claims 1-20 wherein R² is H.

22. The compound of any one of claims 1-21 wherein R³ is H.

23. The compound of claim 1 which is: 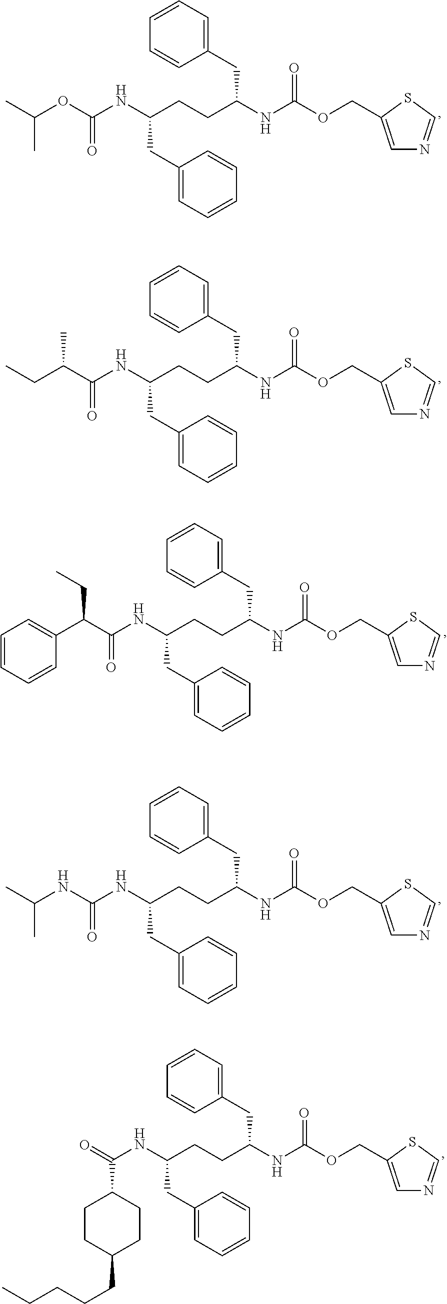 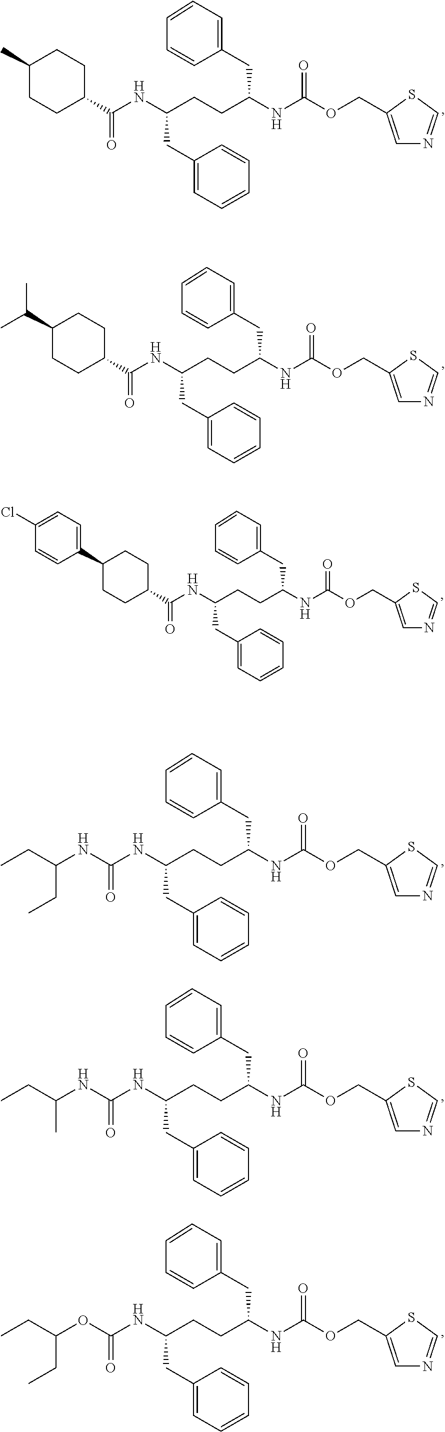 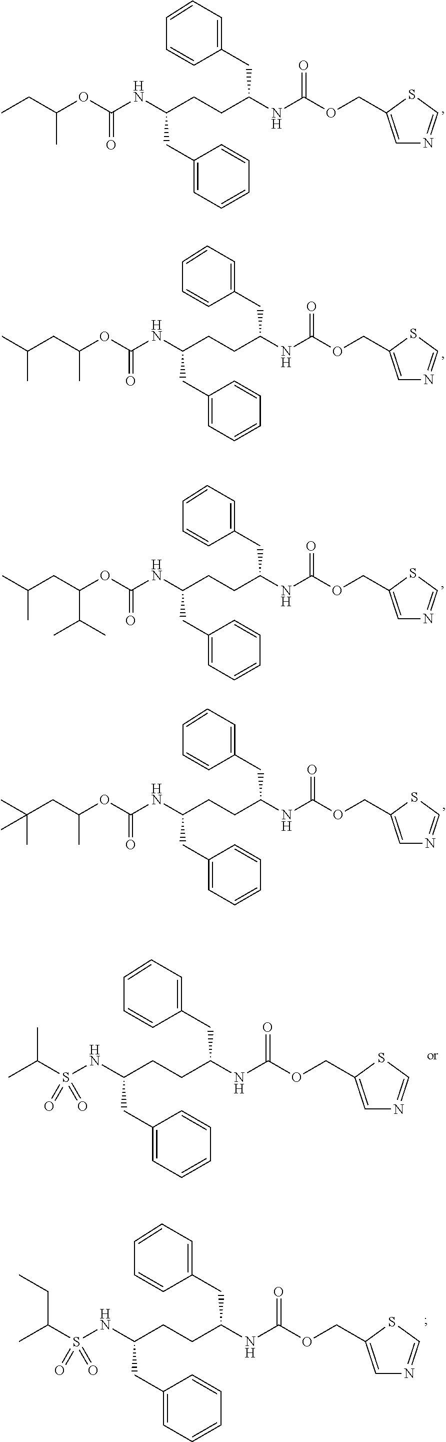
or a salt thereof.

24. A pharmaceutical composition comprising a compound of formula I of as described in any one of claims 1-23, or a pharmaceutically acceptable salt thereof, and a pharmaceutically acceptable carrier or excipient.

25. The pharmaceutical composition of claim 24, further comprising one or more therapeutic agents metabolized by cyctochrome P450 monooxygenase.

26. The pharmaceutical composition of claim 25 wherein the therapeutic agents metabolized by cyctochrome P450 are selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, entry inhibitors, gp120 inhibitors, G6PD and NADH-oxidase inhibitors, CCR5 inhibitors, other drugs for treating HIV, interferons, ribavirin analogs, NS5b polymerase inhibitors, NS3 protease inhibitors, alpha-glucosidase 1 inhibitors, hepatoprotectants, non-nucleoside inhibitors of HCV and other drugs for treating HCV.

27. A method for improving the pharmacokinetics or increasing blood plasma levels of one or more therapeutic agents metabolized by cytochrome P450 monooxygenase, comprising co-administering to a patient treated with one or more therapeutic agents metabolized by cytochrome P450 monooxygenase, a pharmacokinetic improving or blood plasma level increasing effective amount of a compound of formula I as described in any one of claims 1-23, or a pharmaceutically acceptable salt thereof.

28. A compound of formula I as described in any one of claims 1-23, or a pharmaceutically acceptable salt thereof for use in medical therapy.

29. The use of a compound of formula I as described in any one of claims 1-23, or a pharmaceutically acceptable salt thereof, for the manufacture of a medicament useful for improving the pharmacokinetics of a therapeutic agent which is metabolized by cytochrome P450 monooxygenase or increasing the blood plasma levels of a therapeutic agent which is metabolized by cytochrome P450 monooxygenase.

30. The use of claim 29 wherein the therapeutic agent metabolized by cytochrome P450 monooxygenase is selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, entry inhibitors, gp120 inhibitors, G6PD and NADH-oxidase inhibitors, CCR5 inhibitors, other drugs for treating HIV, interferons, ribavirin analogs, NS5b polymerase inhibitors, NS3 protease inhibitors, alpha-glucosidase 1 inhibitors, hepatoprotectants, non-nucleoside inhibitors of HCV and other drugs for treating HCV, and combinations thereof.
